(19) United States
(12) Patent Application Publication
(10) Pub. No.: US 2004/0105853 A1
(43) Pub. Date: Jun. 3, 2004

(54) Regulation of human epithin-like serine-protease

(76) Inventor: Yonghong Xiao, Cambridge, MA (US)

Correspondence Address: BANNER & WITCOFF, 1001 G STREET N W, SUITE 1100, WASHINGTON, DC, 20001, US

(21) Appl. No.: 10/297,987

(22) PCT Filed: Jun. 11, 2001
(86) PCT No.: PCT/EP01/06566

(51) Int. Cl. C12Q 1/68; C07H 21/04; A61K 38/48; C12N 9/64

(52) U.S. Cl. 424/94.64; 435/6; 435/69.1; 435/226; 435/320.1; 435/325; 536/23.2

(57) ABSTRACT

Reagents which regulate human epithin-like serine protease activity and reagents which bind to human epithin-like serine protease gene products can be used to regulate extracellular matrix degradation. Such regulation is particularly useful for treating metastasis of malignant cells, tumor angiogenesis, inflammation, atherosclerosis, neurodegenerative diseases, COPD, and pathogenic infections.

### TECHNICAL FIELD OF THE INVENTION

The invention relates to the area of regulation human epithin-like serine protease activity to provide therapeutic effects.

### BACKGROUND OF THE INVENTION

Metastasizing cancer cells invade the extracellular matrix using plasma membrane protrusions that contact and dissolve the matrix with proteases. Agents which inhibit such protease activity can be used to suppress metastases. Proteases also are expressed during development, when degradation of the extracellular matrix is desired. In cases where appropriate extracellular matrix degradation does not occur, supplying a molecule with a protease activity can provide the necessary enzymatic activity. Thus, there is a need in the art for identifying new proteases and methods of regulating extracellular matrix degradation.

### SUMMARY OF THE INVENTION

It is an object of the invention to provide reagents and methods of regulating human epithin-like serine protease. These and other objects of the invention are provided by one or more of the embodiments described below.

One embodiment of the invention is a epithin-like serine protease polypeptide comprising an amino acid sequence selected from the group consisting of:

amino acid sequences which are at least about 50% identical to the amino acid sequence shown in SEQ ID NO: 10;

the amino acid sequence shown in SEQ ID NO: 10;

amino acid sequences which are at least about 50% identical to the amino acid sequence shown in SEQ ID NO: 24;

the amino acid sequence shown in SEQ ID NO: 24;

amino acid sequences which are at least about 50% identical to the amino acid sequence shown in SEQ ID NO: 27; and

the amino acid sequence shown in SEQ ID NO: 27.

Yet another embodiment of the invention is a method of screening for agents which decrease extracellular matrix degradation. A test compound is contacted with a epithin-like serine protease polypeptide comprising an amino acid sequence selected from the group consisting of:

amino acid sequences which are at least about 50% identical to the amino acid sequence shown in SEQ ID NO: 10;

the amino acid sequence shown in SEQ ID NO: 10;

amino acid sequences which are at least about 50% identical to the amino acid sequence shown in SEQ ID NO: 24;

the amino acid sequence shown in SEQ ID NO: 24;

amino acid sequences which are at least about 50% identical to the amino acid sequence shown in SEQ ID NO: 27; and

the amino acid sequence shown in SEQ ID NO: 27.

Binding between the test compound and the epithin-like serine protease polypeptide is detected. A test compound which binds to the epithin-like serine protease polypeptide is thereby identified as a potential agent for decreasing extracellular matrix degradation. The agent can work by decreasing the activity of the epithin-like serine protease.

Another embodiment of the invention is a method of screening for agents which decrease extracellular matrix degradation. A test compound is contacted with a polynucleotide encoding a epithin-like serine protease polypeptide, wherein the polynucleotide comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 2;

the nucleotide sequence shown in SEQ ID NO: 2;

nucleotide sequences which are at least about 50% identical to the nucleotide sequence shown in SEQ ID NO: 3;

the nucleotide sequence shown in SEQ ID NO:3;

nucleotide sequences which are at least about 50% identical to the nucleotide sequence shown in SEQ ID NO: 4:

the nucleotide sequence shown in SEQ ID NO: 4;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID NO: 6;

the nucleotide sequence shown in SEQ ID NO: 6;

nucleotide sequences which are at least about 50% identical to the nucleotide sequence shown in SEQ ID NO: 7;

the nucleotide sequence shown in SEQ ID NO: 7;

nucleotide sequences which are at least about 50% identical to the nucleotide sequence shown in SEQ ID NO: 8;

the nucleotide sequence shown in SEQ ID NO: 8;

nucleotide sequences which are at least about 50% identical to the nucleotide sequence shown in SEQ ID NO: 9;

the nucleotide sequence shown in SEQ ID NO: 9;

nucleotide sequences which are at least about 50% identical to the nucleotide sequence shown in SEQ ID NO: 25;

the nucleotide sequence shown in SEQ ID NO: 25;

nucleotide sequences which are at least about 50% identical to the nucleotide sequence shown in SEQ ID NO: 26; and

the nucleotide sequence shown in SEQ ID NO:26.

Binding of the test compound to the polynucleotide is detected. A test compound which binds to the polynucleotide is identified as a potential agent for decreasing extracellular matrix degradation. The agent can work by decreasing the amount of the epithin-like serine protease through interacting with the epithin-like serine protease mRNA.

Another embodiment of the invention is a method of screening for agents which regulate extracellular matrix degradation. A test compound is contacted with a epithin-like serine protease polypeptide comprising an amino acid sequence selected from the group consisting of:

amino acid sequences which are at least about 50% identical to the amino acid sequence shown in SEQ ID NO: 10;

the amino acid sequence shown in SEQ ID NO: 10;

amino acid sequences which are at least about 50% identical to the amino acid sequence shown in SEQ ID NO: 24;

the amino acid sequence shown in SEQ ID NO: 24;

amino acid sequences which are at least about 50% identical to the amino acid sequence shown in SEQ ID NO: 27; and

the amino acid sequence shown in SEQ ID NO: 27.

A epithin-like serine protease activity of the polypeptide is detected. A test compound which increases epithin-like serine protease activity of the polypeptide relative to epithin-like serine protease activity in the absence of the test compound is thereby identified as a potential agent for increasing extracellular matrix degradation. A test compound which decreases epithin-like serine protease activity of the polypeptide relative to epithin-like serine protease activity in the absence of the test compound is thereby identified as a potential agent for decreasing extracellular matrix degradation.

Even another embodiment of the invention is a method of screening for agents which decrease extracellular matrix degradation. A test compound is contacted with a epithin-like serine protease product of a polynucleotide which comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 2;

the nucleotide sequence shown in SEQ ID NO: 2;

nucleotide sequences which are at least about 50% identical to the nucleotide sequence shown in SEQ ID NO: 3;

the nucleotide sequence shown in SEQ ID NO: 3;

nucleotide sequences which are at least about 50% identical to the nucleotide sequence shown in SEQ ID NO: 4;

the nucleotide sequence shown in SEQ ID NO: 4;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID NO: 6;

the nucleotide sequence shown in SEQ ID NO:6;

nucleotide sequences which are at least about 50% identical to the nucleotide sequence shown in SEQ ID NO: 7;

the nucleotide sequence shown in SEQ ID NO: 7;

nucleotide sequences which are at least about 50% identical to the nucleotide sequence shown in SEQ ID NO: 8;

the nucleotide sequence shown in SEQ ID NO: 8;

nucleotide sequences which are at least about 50% identical to the nucleotide sequence shown in SEQ ID NO: 9;

the nucleotide sequence shown in SEQ ID NO: 9;

nucleotide sequences which are at least about 50% identical to the nucleotide sequence shown in SEQ ID NO: 25:

the nucleotide sequence shown in SEQ ID NO: 25;

nucleotide sequences which are at least about 50% identical to the nucleotide sequence shown in SEQ ID NO: 26; and

the nucleotide sequence shown in SEQ ID NO:26.

Binding of the test compound to the epithin-like serine protease product is detected. A test compound which binds to the epithin-like serine protease product is thereby identified as a potential agent for decreasing extracellular matrix degradation.

Still another embodiment of the invention is a method of reducing extracellular matrix degradation. A cell is contacted with a reagent which specifically binds to a polynucleotide encoding a epithin-like serine protease polypeptide or the product encoded by the polynucleotide, wherein the polynucleotide comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 2;

the nucleotide sequence shown in SEQ ID NO: 2;

nucleotide sequences which are at least about 50% identical to the nucleotide sequence shown in SEQ ID NO: 3;

the nucleotide sequence shown in SEQ ID NO:3;

nucleotide sequences which are at least about 50% identical to the nucleotide sequence shown in SEQ ID NO: 4;

the nucleotide sequence shown in SEQ ID NO: 4;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID NO: 6;

the nucleotide sequence shown in SEQ ID NO:6;

nucleotide sequences which are at least about 50% identical to the nucleotide sequence shown in SEQ ID NO: 7;

the nucleotide sequence shown in SEQ ID NO: 7;

nucleotide sequences which are at least about 50% identical to the nucleotide sequence shown in SEQ ID NO: 8;

the nucleotide sequence shown in SEQ ID NO: 8;

nucleotide sequences which are at least about 50% identical to the nucleotide sequence shown in SEQ ID NO: 9;

the nucleotide sequence shown in SEQ ID NO:9; nucleotide sequences which are at least about 50% identical to the nucleotide sequence shown in SEQ ID NO: 25;

the nucleotide sequence shown in SEQ ID NO: 25;

nucleotide sequences which are at least about 50% identical to the nucleotide sequence shown in SEQ ID NO: 26; and

the nucleotide sequence shown in SEQ ID NO:26.

Epithin-like serine protease activity in the cell is thereby decreased.

The invention thus provides reagents and methods for regulating human epithin-like serine protease activity which can be used inter alia, to suppress metastatic activity of malignant cells, to treat COPD, and to enhance extracellular matrix degradation during development.

### BRIEF DESCRIPTION OF THE DRAWINGS

FIG. 1 shows the exon/intron structure of the epithin-like serine protease gene as identified by an alignment with the genomic sequence of ACO11522.6

FIG. 2 shows the alignment of epithin-like serine protease polypeptide as shown in SEQ ID NO: 10 with the protein identified by SwissProt Accession No. P56677 as mouse epithin (SEQ ID NO: 11).

FIG. 3 shows the BLOCKS search results.

FIG. 4 shows the relative expression of epithin-like serine protease in various human tissues.

FIG. 5 shows the relative expression of epithin-like serine protease in various human respiratory tissues and cells.

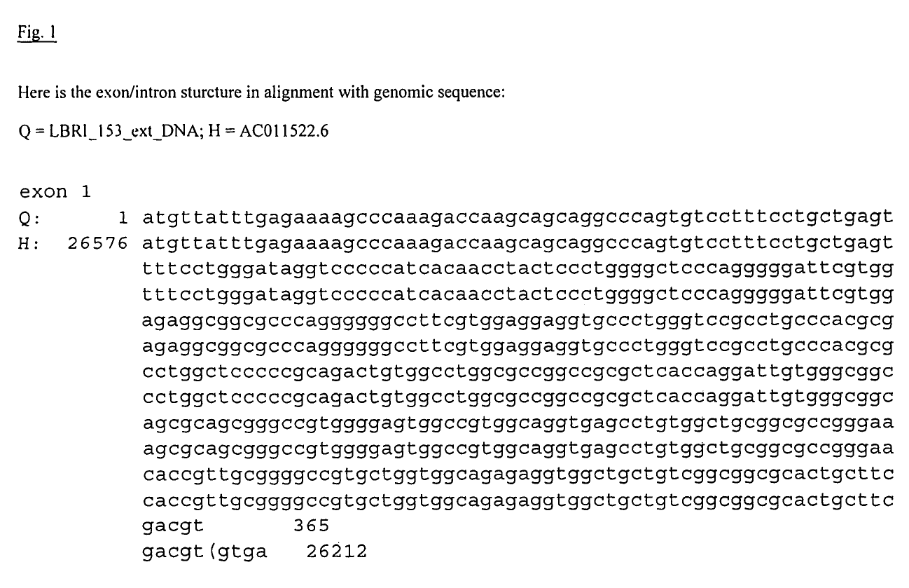

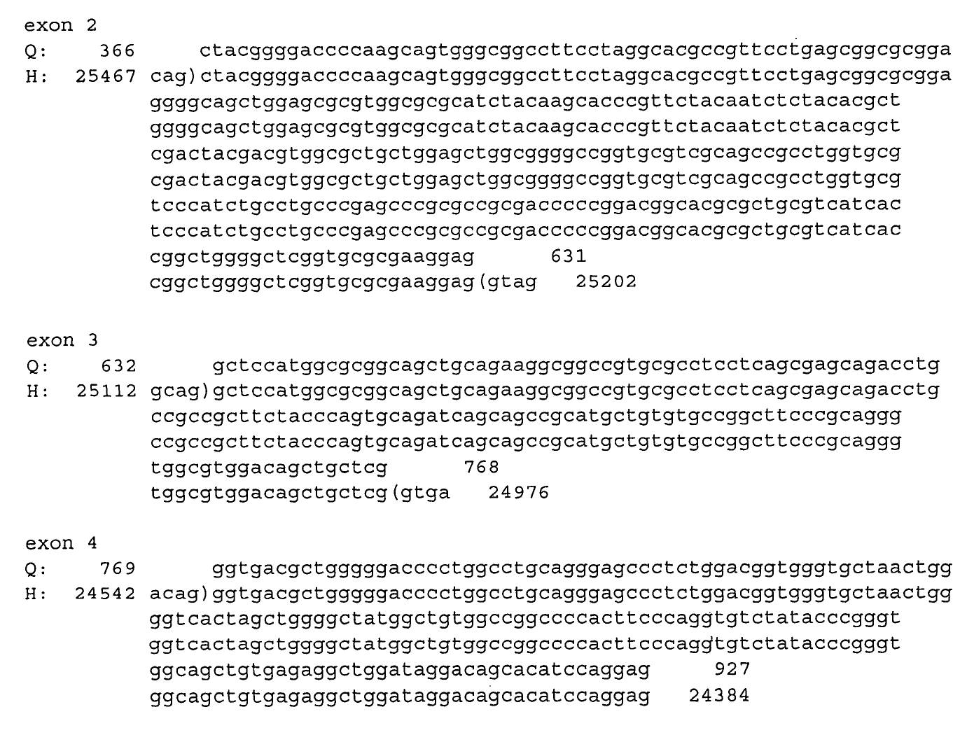

### DETAILED DESCRIPTION OF THE INVENTION

The invention relates to an isolated polynucleotide encoding a epithin-like serine protease polypeptide and being selected from the group consisting of:

a) a polynucleotide encoding a epithin-like serine protease polypeptide comprising an amino acid sequence selected from the group consisting of:

amino acid sequences which are at least about 50% identical to

the amino acid sequence shown in SEQ ID NO: 10;

the amino acid sequence shown in SEQ ID NO: 10;

amino acid sequences which are at least about 50% identical to the amino acid sequence shown in SEQ ID NO: 24;

the amino acid sequence shown in SEQ ID NO: 24;

amino acid sequences which are at least about 50% identical to the amino acid sequence shown in SEQ ID NO: 27; and

the amino acid sequence shown in SEQ ID NO:27.

b) a polynucleotide comprising the sequence of SEQ ID NOS: 10, 25 or 26;

c) a polynucleotide which hybridizes under stringent conditions to a polynucleotide specified in (a) and (b);

d) a polynucleotide the sequence of which deviates from the polynucleotide sequences specified in (a) to (c) due to the degeneration of the genetic code; and

e) a polynucleotide which represents a fragment, derivative or allelic variation of a polynucleotide sequence specified in (a) to (d).

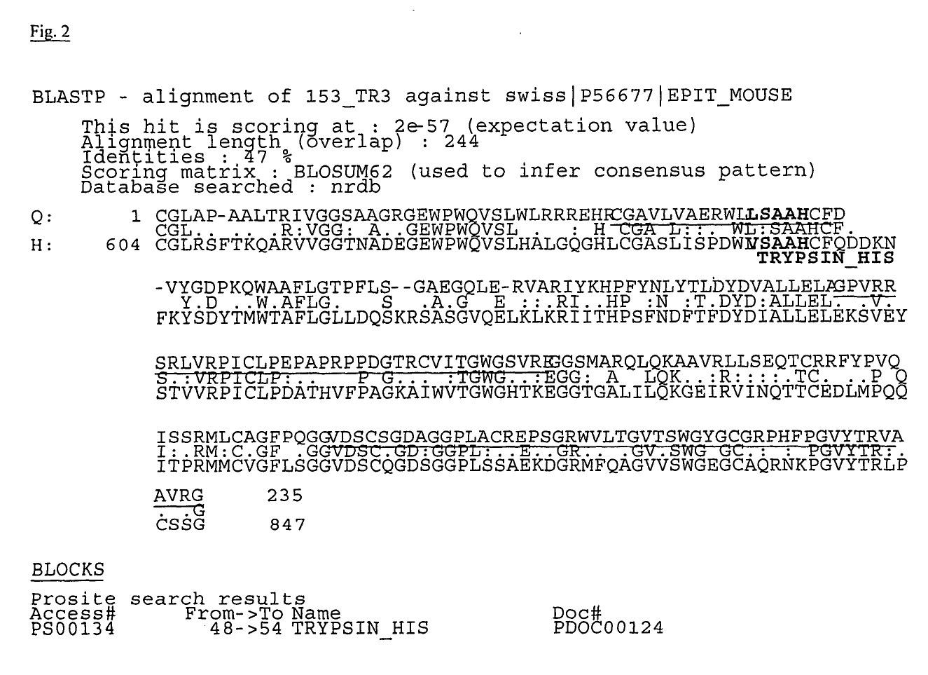

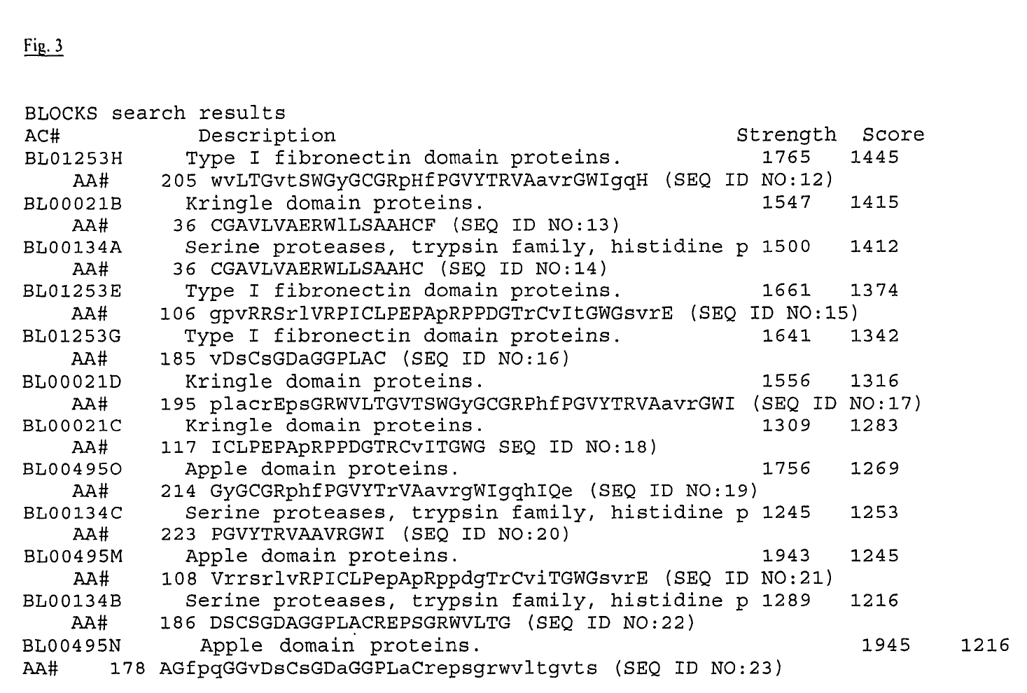

Furthermore, it has been discovered by the present applicant that regulators of a epithin-like serine protease, particularly a human epithin-like serine protease can be used to regulate degradation of the extracellular matrix. Human epithin-like serine protease as shown in SEQ ID NO: 10 is 47% identical to the protein identified by SwissProt Accession No. P56677 (SEQ ID NO: 11) as mouse epithin over a 244 amino acid overlap (FIG. 2). The results of Prosite and BLOCKS searches are shown in FIGS. 2 and 3, respectively. An extended amino acid sequence of human epithin-like serine protease is shown in SEQ ID NO: 24.

Human epithin-like serine protease is therefore expected to be useful for the same purposes as previously identified serine proteases. Human epithin-like serine protease is particularly useful for treating cancer and COPD.

Polypeptides

Epithin-like serine protease polypeptides according to the invention comprise at least 6, 8, 10, 15, 25, 50, 75, 100, 125, 150, 175, 200, 225, or 243 contiguous amino acids selected from SEQ ID NO: 10 or at least 6, 8, 10, 15, 25, 50, 75, 100, 125, 150, 175, 200, 225, 250, 275, 300, 350, 400, 450, 500, or 549 contiguous amino acids selected form SEQ ID NO:24 or 27 or from biologically active variants thereof, as defined below. An epithin-like serine protease polypeptide of the invention therefore can be a portion of an epithin-like serine protease molecule, a full-length epithin-like serine protease molecule, or a fusion protein comprising all or a portion of an epithin-like serine protease molecule.

Biologically Active Variants

Epithin-like serine protease variants which are biologically active, i.e., retain an epithin-like serine protease activity, also are epithin-like serine protease polypeptides. Preferably, naturally or non-naturally occurring epithin-like serine protease variants have amino acid sequences which are at least about 50, preferably about 75, 90, 96, or 98% identical to an amino acid sequence shown in SEQ ID NO:10, 24 or 27. Percent identity between a putative epithin-like serine protease variant and an amino acid sequence of SEQ ID NO:10 is determined with the Needleman/Wunsch algorithm (Needleman and Wunsch, J. Mol. Biol. 48; 443-453, 1970) using a Blosum62 matrix with a gap creation penalty of 8 and a gap extension penalty of 2 (S. Henikoff and J. G. Henikoff, Proc. Natl. Acad. Sci. USA 89:10915-10919, 1992).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity can be found using computer programs well known in the art, such as DNASTAR software. Whether an amino acid change results in a biologically active epithin-like serine protease polypeptide can readily be determined by assaying for fibronectin binding or for epithin-like serine protease activity, as is known in the art and described, for example, in Example 2.

Fusion Proteins

Fusion proteins are useful for generating antibodies against epithin-like serine protease amino acid sequences and for use in various assay systems. For example, fusion proteins can be used to identify proteins which interact with portions of an epithin-like serine protease polypeptide, including its active site and fibronectin domains. Methods such as protein affinity chromatography or library-based assays for protein-protein interactions, such as the yeast two-hybrid or phage display systems, can be used for this purpose. Such methods are well known in the art and also can be used as drug screens.

An epithin-like serine protease fusion protein comprises two protein segments fused together by means of a peptide bond. For example, the first protein segment can comprise at least 6, 8, 10, 15, 25, 50, 75, 100, 125, 150, 175, 200, 225, or 243 contiguous amino acids selected from SEQ ID NO:10 or at least 6, 8, 10, 15, 25, 50, 75, 100, 125, 150, 175, 200, 225, 250, 275, 300, 350, 400, 450, 500, or 549 contiguous amino acids selected form SEQ ID NO:24 or 27 or from biologically active variants thereof. Preferably, a fusion protein comprises the active site of the protease and/or one or more of the functional domains identified in FIGS. 2 and 3. The first protein segment also can comprise full-length epithin-like serine protease.

The second protein segment can be a full-length protein or a protein fragment or polypeptide. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the epithin-like serine protease polypeptide-encoding sequence and the heterologous protein sequence, so that the epithin-like serine protease polypeptide can be cleaved and purified away from the heterologous moiety.

A fusion protein can be synthesized chemically, as is known in the art. Preferably, a fusion protein is produced by covalently linking two protein segments or by standard procedures in the art of molecular biology. Recombinant DNA methods can be used to prepare fusion proteins, for example, by making a DNA construct which comprises epithin-like serine protease coding sequences disclosed herein in proper reading frame with nucleotides encoding the second protein segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of human epithin-like serine protease can be obtained using epithin-like serine protease polynucleotides (described below) to make suitable probes or primers to screening cDNA expression libraries from other species, such as mice, monkeys, or yeast, identifying cDNAs which encode homologs of epithin-like serine protease, and expressing the cDNAs as is known in the art.

Polynucleotides

An epithin-like serine protease polynucleotide can be single- or double-stranded and comprises a coding sequence or the complement of a coding sequence for an epithin-like serine protease polypeptide. Coding sequences for the epithin-like serine protease are shown in SEQ ID NOS: 10, 25 and 26.

Degenerate nucleotide sequences encoding human epithin-like serine protease polypeptides, as well as homologous nucleotide sequences which are at least about 50, preferably about 75, 90, 96, or 98% identical to the epithin-like serine protease coding sequences shown in SEQ ID NOS: 25 and 26 also are epithin-like serine protease polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of epithin-like serine protease polynucleotides which encode biologically active epithin-like serine protease polypeptides also are epithin-like serine protease polynucleotides.

Identification of Variants and Homologs

Variants and homologs of the epithin-like serine protease polynucleotides disclosed above also are epithin-like serine protease polynucleotides. Typically, homologous epithin-like serine protease polynucleotide sequences can be identified by hybridization of candidate polynucleotides to known epithin-like serine protease polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the epithin-like serine protease polynucleotides disclosed herein can be identified by making suitable probes or primers and screening cDNA expression libraries from other species, such as mice, monkeys, or yeast. Human variants of epithin-like serine protease polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human epithin-like serine protease polynucleotides or epithin-like serine protease polynucleotides of other species can therefore be identified, for example by hybridizing a putative homologous epithin-like serine protease polynucleotide with a polynucleotide having a nucleotide sequence of SEQ ID NO:25 or 26 to form a test hybrid. The melting temperature of the test hybrid is compared with the melting temperature of a hybrid comprising epithin-like serine protease polynucleotides having perfectly complementary nucleotide sequences, and the number or percent of basepair mismatches within the test hybrid is calculated.

Nucleotide sequences which hybridize to epithin-like serine protease polynucleotides or their complements following stringent hybridization and/or wash conditions are also epithin-like serine protease polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between an epithin-like serine protease polynucleotide having a coding sequence disclosed herein and a polynucleotide sequence which is at least about 50, preferably about 75, 90, 96, or 98% identical to that nucleotide sequence can be calculated, for example, using the equation of Bolton and McCarthy, Proc. Natl. Acad. Sci. U.S.A. 48, 1390 (1962):

Tₘ=81.5°C.−16.6(log₁₀ [Na⁺])+0.41(% G+C)−0.63(% formamide)−600/l),

where l=the length of the hybrid in basepairs.

Stringent wash conditions include, for example, 4×SSC at 65° C, or 50% formamide, 4×SSC at 42° C, or 0.5×SSC, 0.1% SDS at 65° C. Highly stringent wash conditions include, for example, 0.2×SSC at 65° C.

Preparation of Polynucleotides

A naturally occurring epithin-like serine protease polynucleotide can be isolated free of other cellular components such as membrane components, proteins, and lipids. Polynucleotides can be made by a cell and isolated using standard nucleic acid purification techniques, synthesized using an amplification technique, such as the polymerase chain reaction (PCR), or synthesized using an automatic synthesizer. Methods for isolating polynucleotides are routine and are known in the art. Any such technique for obtaining a polynucleotide can be used to obtain isolated epithin-like serine protease polynucleotides. For example, restriction enzymes and probes can be used to isolate polynucleotide fragments which comprise epithin-like serine protease nucleotide sequences. Isolated polynucleotides are in preparations which are free or at least 70, 80, or 90% free of other molecules.

Epithin-like serine protease cDNA molecules can be made with standard molecular biology techniques, using epithin-like serine protease mRNA as a template. Epithin-like serine protease cDNA molecules can thereafter be replicated using molecular biology techniques known in the art and disclosed in manuals such as Sambrook et al. (1989). An amplification technique, such as PCR, can be used to obtain additional copies of epithin-like serine protease polynucleotides, using either human genomic DNA or cDNA as a template.

Alternatively, synthetic chemistry techniques can be used to synthesize epithin-like serine protease polynucleotides. The degeneracy of the genetic code allows alternate nucleotide sequences to be synthesized which will encode an epithin-like serine protease polypeptide having, for example, the amino acid sequence shown in SEQ ID NO:10, 24 or 27 or a biologically active variant thereof.

Obtaining Full-Length Polynucleotides

The partial sequence of SEQ ID NOS:25 or 26 or their complements can be used to identify the corresponding full length gene from which they were derived. The partial sequences can be nick-translated or end-labeled with ³²P using polynucleotide kinase using labeling methods known to those with skill in the art (BASIC METHODS IN MOLECULAR BIOLOGY, Davis et al., eds., Elsevier Press. N.Y., 1986). A lambda library prepared from human tissue can be directly screened with the labeled sequences of interest or the library can be converted en masse to pBluescript (Stratagene Cloning Systems, La Jolla, Calif. 92037) to facilitate bacterial colony screening (see Sambrook et al., 1989, pg. 1.20).

Both methods are well known in the art. Briefly, filters with bacterial colonies containing the library in pBluescript or bacterial lawns containing lambda plaques are denatured, and the DNA is fixed to the filters. The filters are hybridized with the labeled probe using hybridization conditions described by Davis et al., 1986. The partial sequences, cloned into lambda or pBluescript, can be used as positive controls to assess background binding and to adjust the hybridization and washing stringencies necessary for accurate clone identification. The resulting autoradiograms are compared to duplicate plates of colonies or plaques; each exposed spot corresponds to a positive colony or plaque. The colonies or plaques are selected and expanded, and the DNA is isolated from the colonies for further analysis and sequencing.

Positive cDNA clones are analyzed to determine the amount of additional sequence they contain using PCR with one primer from the partial sequence and the other primer from the vector. Clones with a larger vector-insert PCR product than the original partial sequence are analyzed by restriction digestion and DNA sequencing to determine whether they contain an insert of the same size or similar as the mRNA size determined from Northern blot Analysis.

Once one or more overlapping cDNA clones are identified, the complete sequence of the clones can be determined, for example after exonuclease III digestion (McCombie et al., Methods 3, 33-40, 1991). A series of deletion clones are generated, each of which is sequenced. The resulting overlapping sequences are assembled into a single contiguous sequence of high redundancy (usually three to five overlapping sequences at each nucleotide position), resulting in a highly accurate final sequence.

Various PCR-based methods can be used to extend the nucleic acid sequences encoding the disclosed portions of human epithin-like serine protease to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations are used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991. Additionally, PCR, nested primers, and PROMOTERFINDER libraries (CLONTECH, Palo Alto, Calif.) can be used to walk genomic DNA. This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

Obtaining Polypeptides

Epithin-like serine protease polypeptides can be obtained, for example, by purification from human cells, by expression of epithin-like serine protease polynucleotides, or by direct chemical synthesis.

Protein Purification

Epithin-like serine protease polypeptides can be purified from cells, such as primary tumor cells, metastatic cells, or cancer cell lines (e.g., colon cancer cell lines HCT116, DLD1, HT29, Caco2, SW837, SW480, and RKO, breast cancer cell lines 21-PT, 21-MT, MDA-468, SK-BR3, and BT-474, the A549 lung cancer cell line, or the H392 glioblastoma cell line), as well as cells transfected with an epithin-like serine protease expression construct. Fetal liver and spleen, clear cell type kidney tumors, ovarian tumors, lung carcinoid tissue, chronic lymphatic leukemia, and pectoral muscle are especially useful sources of epithin-like serine protease polypeptides. A purified epithin-like serine protease polypeptide is separated from other compounds which normally associate with the epithin-like serine protease polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified epithin-like serine protease polypeptides is at least 80% pure; preferably, the preparations are 90%, 95%, or 99% pure. Purity of the preparations can be assessed by any means known in the art, such as SDS-polyacrylamide gel electrophoresis. Enzymatic activity of the purified preparations can be assayed, for example, as described in Example 2.

Expression of Polynucleotides

To express an epithin-like serine protease polypeptide, an epithin-like serine protease polynucleotide can be inserted into an expression vector which contains the necessary elements for the transcription and translation of the inserted coding sequence. Methods which are well known to those skilled in the art can be used to construct expression vectors containing sequences encoding epithin-like serine protease polypeptides and appropriate transcriptional and translational control elements. These methods include in vitro recombinant DNA techniques, synthetic techniques, and in vivo genetic recombination. Such techniques are described, for example, in Sambrook et al. (1989) and Ausubel et al., CURRENT PROTOCOLS IN MOLECULAR BIOLOGY, John Wiley & Sons, New York, N.Y, 1989.

A variety of expression vector/host systems can be utilized to contain and express sequences encoding an epithin-like serine protease polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding an epithin-like serine protease polypeptide, vectors based on SV40 or EBV can be used with an appropriate selectable marker.

Bacterial and Yeast Expression Systems

In bacterial systems, a number of expression vectors can be selected depending upon the use intended for the epithin-like serine protease polypeptide. For example, when a large quantity of an epithin-like serine protease polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene), in which the sequence encoding the epithin-like serine protease polypeptide can be ligated into the vector in frame with sequences for the amino-terminal Met and the subsequent 7 residues of β-galactosidase so that a hybrid protein is produced. pIN vectors (Van Heeke & Schuster, J. Biol. Chem. 264, 5503-5509, 1989 or pGEX vectors (Promega, Madison, Wis.) can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or Factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding epithin-like serine protease polypeptides can be driven by any of a number of promoters. For example, viral promoters such as the 35S and 19S promoters of CaMV can be used alone or in combination with the omega leader sequence from TMV (Takamatsu EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (see, for example, Hobbs or Murray, in MCGRAW HILL YEARBOOK OF SCIENCE AND TECHNOLOGY, McGraw Hill, New York, N.Y., pp. 191-196, 1992).

An insect system also can be used to express an epithin-like serine protease polypeptide. For example, in one such system Autographa californica nuclear polyhedrosis virus (AcNPV) is used as a vector to express foreign genes in Spodoptera frugiperda cells or in Trichoplusia larvae. Sequences encoding epithin-like serine protease polypeptides can be cloned into a non-essential region of the virus, such as the polyhedrin gene, and placed under control of the polyhedrin promoter. Successful insertion of epithin-like serine protease polypeptides will render the polyhedrin gene inactive and produce recombinant virus lacking coat protein. The recombinant viruses can then be used to infect, for example, S. frugiperda cells or Trichoplusia larvae in which epithin-like serine protease polypeptides can be expressed (Engelhard et al., Proc. Nat. Acad. Sci. 91, 3224-3227, 1994).

Mammalian Expression Systems

A number of viral-based expression systems can be utilized in mammalian host cells. For example, if an adenovirus is used as an expression vector, sequences encoding epithin-like serine protease polypeptides can be ligated into an adenovirus transcription/translation complex consisting of the late promoter and tripartite leader sequence. Insertion in a non-essential E1 or E3 region of the viral genome can be used to obtain a viable virus which is capable of expressing an epithin-like serine protease polypeptide in infected host cells (Logan & Shenk, Proc. Natl. Acad Sci. 81. 3655-3659, 1984). In addition, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding epithin-like serine protease polypeptides. Such signals include the ATG initiation codon and adjacent sequences. In cases where sequences encoding an epithin-like serine protease polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

Host Cells

A host cell strain can be chosen for its ability to modulate the expression of the inserted sequences or to process an expressed epithin-like serine protease polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function.

Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express epithin-like serine protease polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced epithin-like serine protease sequences. Resistant clones of stably transformed cells can be proliferated using tissue culture techniques appropriate to the cell type.

Any number of selection systems can be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980). Genes which can be employed in tk- or aprt-cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980); npt confers resistance to the aminoglycosides, neomycin and G-418 (Colbere-Garapin et al., J. Mol. Biol. 150, 1-14, 1981); and als and pal confer resistance to chlorsulfuron and phosphinotricin acetyltransferase, respectively (Murray, 1992 supra). Additional selectable genes have been described, for example trpB, which allows cells to utilize indole in place of tryptophan, or hisD, which allows cells to utilize histinol in place of histidine (Hartman & Mulligan, Proc. Natl. Acad. Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression of Polypeptides

Although the presence of marker gene expression suggests that the epithin-like serine protease polynucleotide is also present, its presence and expression may need to be confirmed. For example, if a sequence encoding an epithin-like serine protease polypeptide is inserted within a marker gene sequence, transformed cells containing sequences which encode an epithin-like serine protease polypeptide can be identified by the absence of marker gene function. Alternatively, a marker gene can be placed in tandem with a sequence encoding an epithin-like serine protease polypeptide under the control of a single promoter. Expression of the marker gene in response to induction or selection usually indicates expression of the epithin-like serine protease polynucleotide.

Alternatively, host cells which contain an epithin-like serine protease polynucleotide and which express an epithin-like serine protease polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein.

The presence of a polynucleotide sequence encoding an epithin-like serine protease polypeptide can be detected by DNA-DNA or DNA-RNA hybridization or amplification using probes or fragments or fragments of polynucleotides encoding an epithin-like serine protease polypeptide. Nucleic acid amplification-based assays involve the use of oligonucleotides selected from sequences encoding an epithin-like serine protease polypeptide to detect transformants which contain an epithin-like serine protease polynucleotide.

A variety of protocols for detecting and measuring the expression of an epithin-like serine protease polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on an epithin-like serine protease polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding epithin-like serine protease polypeptides include oligolabeling, nick translation, end-labeling, or PCR amplification using a labeled nucleotide. Alternatively, sequences encoding an epithin-like serine protease polypeptide can be cloned into a vector for the production of an mRNA probe. Such vectors are known in the art, are commercially available, and can be used to synthesize RNA probes in vitro by addition of labeled nucleotides and an appropriate RNA polymerase, such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, fluorescent, chemiluminescent, or chromogenic agents, as well as substrates, cofactors, inhibitors, magnetic particles, and the like.

Expression and Purification of Polypeptides

Host cells transformed with nucleotide sequences encoding an epithin-like serine protease polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode epithin-like serine protease polypeptides can be designed to contain signal sequences which direct secretion of epithin-like serine protease polypeptides through a prokaryotic or eukaryotic cell membrane.

Other constructions can be used to join a sequence encoding an epithin-like serine protease polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor Xa or enterokinase (Invitrogen, San Diego, Calif.) between the purification domain and the epithin-like serine protease polypeptide can be used to facilitate purification. One such expression vector provides for expression of a fusion protein containing an epithin-like serine protease polypeptide and 6 histidine residues preceding a thioredoxin or an enterokinase cleavage site. The histidine residues facilitate purification on IMAC (immobilized metal ion affinity chromatography as described in Porath et al., Prot. Exp. Purif. 3, 263-281, 1992), while the enterokinase cleavage site provides a means for purifying the epithin-like serine protease polypeptide from the fusion protein. Vectors which contain fusion proteins are disclosed in Kroll et al., DNA Cell Biol. 12, 441-453, 1993).

Chemical Synthesis

Sequences encoding an epithin-like serine protease polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, an epithin-like serine protease polypeptide itself can be produced using chemical methods to synthesize its amino acid sequence. For example, epithin-like serine protease polypeptides can be produced by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Various fragments of epithin-like serine protease polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, W H Freeman and Co., New York, N.Y., 1983). The composition of a synthetic epithin-like serine protease polypeptide can be confirmed by amino acid analysis or sequencing (e.g., the Edman degradation procedure; see Creighton, supra). Additionally, any portion of the amino acid sequence of the epithin-like serine protease polypeptide can be altered during direct synthesis and/or combined using chemical methods with sequences from other proteins to produce a variant polypeptide or a fusion protein.

Production of Altered Polypeptides

As will be understood by those of skill in the art, it may be advantageous to produce epithin-like serine protease polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter epithin-like serine protease polypeptide-encoding sequences for a variety of reasons, including modification of the cloning, processing, and/or expression of the gene product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of an epithin-like serine protease polypeptide. “Antibody” as used herein includes intact immunoglobulin molecules, as well as fragments thereof, such as Fab, F(ab′)₂, and Fv, which are capable of binding an epitope of an epithin-like serine protease polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of an epithin-like serine protease polypeptide can be used therapeutically, as well as in immunochemical assays, including but not limited to Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to an epithin-like serine protease polypeptide provides a detection signal at least 5-, 10-, or 20-fold higher than a detection signal provided with other proteins when used in an immunochemical assay. Preferably, antibodies which specifically bind to epithin-like serine protease polypeptides do not detect other proteins in immunochemical assays and can immunoprecipitate an epithin-like serine protease polypeptide from solution.

Epithin-like serine protease polypeptides can be used to immunize a mammal, such as a mouse, rat, rabbit, guinea pig, monkey, or human, to produce polyclonal antibodies. If desired, an epithin-like serine protease polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to an epithin-like serine protease polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 31-42, 1985; Cote et al. Proc. Natl. Acad. Sci. 80, 2026-2030, 1983; Cole et al., Mol. Cell Biol. 62, 109-120. 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, one can produce humanized antibodies using recombinant methods, as described in GB2188638B. Antibodies which specifically bind to an epithin-like serine protease polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to epithin-like serine protease polypeptides. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught in Mallender & Voss, 1994, J. Biol. Chem 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology. Verhaar et al., 1995, Int. J. Cancer 61, 497-501; Nicholls et al., 1993, J. Immunol. Meth. 165, 81-91.

Antibodies which specifically bind to epithin-like serine protease polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies of the invention can be purified by methods well known in the art. For example, antibodies can be affinity purified by passage over a column to which an epithin-like serine protease polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of epithin-like serine protease gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of epithin-like serine protease gene expression can be obtained by designing antisense oligonucleotides which will form duplexes to the control, 5′, or regulatory regions of the epithin-like serine protease gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful duplex formation between an antisense oligonucleotide and the complementary sequence of an epithin-like serine protease polynucleotide. Antisense oligonucleotides which comprise, for example, 2, 3, 4, or 5 or more stretches of contiguous nucleotides which are precisely complementary to an epithin-like serine protease polynucleotide, each separated by a stretch of contiguous nucleotides which are not complementary to adjacent epithin-like serine protease nucleotides, can provide targeting specificity for epithin-like serine protease mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular epithin-like serine protease polynucleotide sequence.

Antisense oligonucleotides can be modified without affecting their ability to hybridize to an epithin-like serine protease polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of an epithin-like serine protease polynucleotide can be used to generate ribozymes which will specifically bind to mRNA transcribed from the epithin-like serine protease polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within an epithin-like serine protease RNA target are initially identified by scanning the RNA molecule for ribozyme cleavage sites which include the following sequences: GUA, GUU, and GUC. Once identified, short RNA sequences of between 15 and 20 ribonucleotides corresponding to the region of the epithin-like serine protease target RNA containing the cleavage site can be evaluated for secondary structural features which may render the target inoperable. The suitability of candidate targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related; thus, upon hybridizing to the epithin-like serine protease target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease epithin-like serine protease expression. Alternatively, if it is desired that the cells stably retain the DNA construct, it can be supplied on a plasmid and maintained as a separate element or integrated into the genome of the cells, as is known in the art. The DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of epithin-like serine protease mRNA occurs only when both a ribozyme and a target gene are induced in the cells.

Differentially Expressed Genes

Described herein are methods for the identification of genes whose products interact with human epithin-like serine protease. Such genes may represent genes that are differentially expressed in disorders including, but not limited to, cancer and COPD. Further, such genes may represent genes that are differentially regulated in response to manipulations relevant to the progression or treatment of such diseases. Additionally, such genes may have a temporally modulated expression, increased or decreased at different stages of tissue or organism development. A differentially expressed gene may also have its expression modulated under control versus experimental conditions. In addition, the human epithin-like serine protease gene or gene product may itself be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

Identification of Differentially Expressed Genes

To identify differentially expressed genes total RNA or, preferably, mRNA is isolated from tissues of interest. For example, RNA samples are obtained from tissues of experimental subjects and from corresponding tissues of control subjects. Any RNA isolation technique that does not select against the isolation of mRNA may be utilized for the purification of such RNA samples. See, for example, Ausubel et al., ed., CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples that represent RNA produced by differentially expressed genes are identified by methods well known to those of skill in the art. They include, for example, differential screening (Tedder et al., Proc. Natl. Acad. Sci. U.S.A. 85, 208-12, 1988), subtractive hybridization (Hedrick et al., Nature 308, 149-53; Lee et al., Proc. Natl. Acad. Sci. U.S.A. 88, 2825, 1984), and differential display (Liang & Pardee, Science 257, 967-71, 1992; U.S. Pat. No. 5,262,311), and microarrays.

The differential expression information may itself suggest relevant methods for the treatment of disorders involving the human epithin-like serine protease. For example, treatment may include a modulation of expression of the differentially expressed genes and/or the gene encoding the human epithin-like serine protease. The differential expression information may indicate whether the expression or activity of the differentially expressed gene or gene product or the human epithin-like serine protease gene or gene product are up-regulated or down-regulated.

Screening Methods

The invention provides methods for identifying modulators, i.e., candidate or test compounds which bind to epithin-like serine protease polypeptides or polynucleotides and/or have a stimulatory or inhibitory effect on, for example, expression or activity of the epithin-like serine protease polypeptide or polynucleotide, so as to regulate degradation of the extracellular matrix. Decreased extracellular matrix degradation is useful for preventing or suppressing malignant cells from metastasizing. Increased extracellular matrix degradation may be desired, for example, in developmental disorders characterized by inappropriately low levels of extracellular matrix degradation or in regeneration.

The invention provides assays for screening test compounds which bind to or modulate the activity of an epithin-like serine protease polypeptide or an epithin-like serine protease polynucleotide. A test compound preferably binds to an epithin-like serine protease polypeptide or polynucleotide. More preferably, a test compound decreases an epithin-like serine protease activity of an epithin-like serine protease polypeptide or expression of an epithin-like serine protease polynucleotide by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem Int. Ed Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to epithin-like serine protease polypeptides or polynucleotides or to affect epithin-like serine protease activity or epithin-like serine protease gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (November 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule which binds to and occupies the active site or a fibronectin domain of the epithin-like serine protease polypeptide, thereby making the active site or fibronectin domain inaccessible to substrate such that normal biological activity is prevented. Examples of such small molecules include, but are not limited to, small peptides or peptide-like molecules. In binding assays, either the test compound or the epithin-like serine protease polypeptide can comprise a detectable label, such as a fluorescent, radioisotopic, chemiluminescent, or enzymatic label, such as horseradish peroxidase, alkaline phosphatase, or luciferase. Detection of a test compound which is bound to the epithin-like serine protease polypeptide can then be accomplished, for example, by direct counting of radioemmission, by scintillation counting, or by determining conversion of an appropriate substrate to a detectable product.

Alternatively, binding of a test compound to an epithin-like serine protease polypeptide can be determined without labeling either of the interactants. For example, a microphysiometer can be used to detect binding of a test compound with a target polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and an epithin-like serine protease polypeptide. (McConnell et al., Science 257, 1906-1912, 1992).

Determining the ability of a test compound to bind to an epithin-like serine protease polypeptide also can be accomplished using a technology such as real-time Bimolecular Interaction Analysis (BIA). Sjolander & Urbaniczky, Anal. Chem. 63, 2338-2345, 1991, and Szabo et al., Curr. Opin. Struct. Biol. 5, 699-705, 1995. BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, an epithin-like serine protease polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent W094/10300), to identify other proteins which bind to or interact with the epithin-like serine protease polypeptide and modulate its activity.

The two-hybrid system is based on the modular nature of most transcription factors, which consist of separable DNA-binding and activation domains. Briefly, the assay utilizes two different DNA constructs. For example, in one construct a polynucleotide encoding an epithin-like serine protease polypeptide is fused to a polynucleotide encoding the DNA binding domain of a known transcription factor (e.g., GAL-4). In the other construct, a DNA sequence that encodes an unidentified protein (“prey” or “sample”) is fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g. lacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the epithin-like serine protease polypeptide.

It may be desirable to immobilize either the epithin-like serine protease polypeptide (or polynucleotide) or the test compound to facilitate separation of bound from unbound forms of one or both of the interactants, as well as to accommodate automation of the assay. Thus, either the epithin-like serine protease polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the epithin-like serine protease polypeptide (or polynucleotide) or test compound to a solid support, including use of covalent and non-covalent linkages, passive absorption, or pairs of binding moieties attached respectively to the polypeptide or test compound and the solid support. Test compounds are preferably bound to the solid support in an array, so that the location of individual test compounds can be tracked. Binding of a test compound to an epithin-like serine protease polypeptide (or polynucleotide) can be accomplished in any vessel suitable for containing the reactants. Examples of such vessels include microtiter plates, test tubes, and microcentrifuge tubes.

In one embodiment, an epithin-like serine protease polypeptide is a fusion protein comprising a domain that allows the epithin-like serine protease polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed epithin-like serine protease polypeptide; the mixture is then incubated under conditions conducive to complex formation (e.g., at physiological conditions for salt and pH). Following incubation, the beads or microtiter plate wells are washed to remove any unbound components.

Binding of the interactants can be determined either directly or indirectly, as described above. Alternatively, the complexes can be dissociated from the solid support before binding is determined.

Other techniques for immobilizing polypeptides or polynucleotides on a solid support also can be used in the screening assays of the invention. For example, either an epithin-like serine protease polypeptide (or polynucleotide) or a test compound can be immobilized utilizing conjugation of biotin and streptavidin. Biotinylated epithin-like serine protease polypeptides or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to an epithin-like serine protease polypeptide polynucleotides, or a test compound, but which do not interfere with a desired binding site, such as the active site or a fibronectin domain of the epithin-like serine protease polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the epithin-like serine protease polypeptide (or polynucleotides) or test compound, enzyme-linked assays which rely on detecting an epithin-like serine protease activity of the epithin-like serine protease polypeptide, and SDS gel electrophoresis under non-reducing conditions.

Screening for test compounds which bind to an epithin-like serine protease polypeptide or polynucleotide also can be carried out in an intact cell. Any cell which comprises an epithin-like serine protease polynucleotide or polypeptide can be used in a cell-based assay system. An epithin-like serine protease polynucleotide can be naturally occurring in the cell or can be introduced using techniques such as those described above. Either a primary culture or an established cell line, including neoplastic cell lines such as the colon cancer cell lines HCT116, DLD1, HT29, Caco2, SW837, SW480, and RKO, breast cancer cell lines 21-PT, 21-MT, MDA-468, SK-BR3, and BT-474, the A549 lung cancer cell line, and the H392 glioblastoma cell line, can be used. An intact cell is contacted with a test compound. Binding of the test compound to an epithin-like serine protease polypeptide or polynucleotide is determined as described above, after lysing the cell to release the epithin-like serine protease polypeptide-test compound complex.

Enzyme Assays

Test compounds can be tested for the ability to increase or decrease an epithin-like serine protease activity of an epithin-like serine protease polypeptide. Epithin-like serine protease activity can be measured, for example, using the method described in Example 2. Epithin-like serine protease activity can be measured after contacting either a purified epithin-like serine protease polypeptide, a cell extract, or an intact cell with a test compound. A test compound which decreases epithin-like serine protease activity by at least about 10, preferably about 50, more preferably about 75, 90, or 100% is identified as a potential therapeutic agent for decreasing extracellular matrix degradation. A test compound which increases epithin-like serine protease activity by at least about 10, preferably about 50, more preferably about 75, 90, or 100% is identified as a potential therapeutic agent for increasing extracellular matrix degradation.

Gene Expression

In another embodiment, test compounds which increase or decrease epithin-like serine protease gene expression are identified. An epithin-like serine protease polynucleotide is contacted with a test compound, and the expression of an RNA or polypeptide product of the epithin-like serine protease polynucleotide is determined. The level of expression of epithin-like serine protease mRNA or polypeptide in the presence of the test compound is compared to the level of expression of epithin-like serine protease mRNA or polypeptide in the absence of the test compound. The test compound can then be identified as a modulator of expression based on this comparison. For example, when expression of epithin-like serine protease mRNA or polypeptide is greater in the presence of the test compound than in its absence, the test compound is identified as a stimulator or enhancer of epithin-like serine protease mRNA or polypeptide is less expression. Alternatively, when expression of the mRNA or protein is less in the presence of the test compound than in its absence, the test compound is identified as an inhibitor of epithin-like serine protease mRNA or polypeptide expression.

The level of epithin-like serine protease mRNA or polypeptide expression in the cells can be determined by methods well known in the art for detecting mRNA or protein. Either qualitative or quantitative methods can be used. The presence of polypeptide products of an epithin-like serine protease polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into an epithin-like serine protease polypeptide.

Such screening can be carried out either in a cell-free assay system or in an intact cell. Any cell which expresses an epithin-like serine protease polynucleotide can be used in a cell-based assay system. The epithin-like serine protease polynucleotide can be naturally occurring in the cell or can be introduced using techniques such as those described above. Either a primary culture or an established cell line, including neoplastic cell lines such as the colon cancer cell lines HCT116, DLD1, HT29, Caco2, SW837, SW480, and RKO, breast cancer cell lines 21-PT, 21-MT, MDA-468, SK-BR3, and BT-474, the A549 lung cancer cell line, and the H392 glioblastoma cell line, can be used.

Pharmaceutical Compositions

The invention also provides pharmaceutical compositions which can be administered to a patient to achieve a therapeutic effect. Pharmaceutical compositions of the invention can comprise an epithin-like serine protease polypeptide, epithin-like serine protease polynucleotide, antibodies which specifically bind to an epithin-like serine protease polypeptide, or mimetics, agonists, antagonists, or inhibitors of an epithin-like serine protease polypeptide. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid ester, such as ethyl oleate or triglycerides, or liposomes. Non-lipid polycationic amino polymers also can be used for delivery.

Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

1. Tumor Cell Invasion and Metastasis. Cancer is a disease fundamentally caused by oncogenic cellular transformation. There are several hallmarks of transformed cells that distinguish them from their normal counterparts and underlie the pathophysiology of cancer. These include uncontrolled cellular proliferation, unresponsiveness to normal death-inducing signals (immortalization), increased cellular motility and invasiveness, increased ability to recruit blood supply through induction of new blood vessel formation (angiogenesis), genetic instability, and dysregulated gene expression. Various combinations of these aberrant physiologies, along with the acquisition of drug-resistance frequently lead to an intractable disease state in which organ failure and patient death ultimately ensue.

Most standard cancer therapies target cellular proliferation and rely on the differential proliferative capacities between transformed and normal cells for their efficacy. This approach is hindered by the facts that several important normal cell types are also highly proliferative and that cancer cells frequently become resistant to these agents. Thus, the therapeutic indices for traditional anti-cancer therapies rarely exceed 2.0.

The advent of genomics-driven molecular target identification has opened up the possibility of identifying new cancer-specific targets for therapeutic intervention that will provide safer, more effective treatments for cancer patients. Thus, newly discovered tumor-associated genes and their products can be tested for their role(s) in disease and used as tools to discover and develop innovative therapies. Genes playing important roles in any of the physiological processes outlined above can be characterized as cancer targets.

Genes or gene fragments identified through genomics can readily be expressed in one or more heterologous expression systems to produce functional recombinant proteins. These proteins are characterized in vitro for their biochemical properties and then used as tools in high-throughput molecular screening programs to identify chemical modulators of their biochemical activities. Agonists and/or antagonists of target protein activity can be identified in this manner and subsequently tested in cellular and in vivo disease models for anti-cancer activity. Optimization of lead compounds with iterative testing in biological models and detailed pharmacokinetic and toxicological analyses form the basis for drug development and subsequent testing in humans.

The human epithin-like serine protease gene provides a therapeutic target for decreasing extracellular matrix degradation, in particular for treating or preventing metastatic cancer. For example, blocking a fibronectin domain of human ephrin-like serine protease can suppress or prevent migration or metastasis of tumor cells in response to fibronectin (9, 10). Cancers whose metastasis can be suppressed according to the invention include adenocarcinoma, melanoma, cancers of the adrenal gland, bladder, bone, breast, cervix, gall bladder, liver, lung, ovary, pancreas, prostate, testis, and uterus. Circulating tumor cells arrested in the capillary beds of different organs must invade the endothelial cell lining and degrade its underlying basement membrane (BM) in order to invade into the extravascular tissue(s) where they establish metastasis (1, 2). Metastatic tumor cells often attach at or near the intercellular junctions between adjacent endothelial cells. Such attachment of the metastatic cells is followed by rupture of the junctions, retraction of the endothelial cell borders and migration through the breach in the endothelium toward the exposed underlying BM (1, 11).

Once located between endothelial cells and the BM, the invading cells must degrade the subendothelial glycoproteins and proteoglycans of the BM in order to migrate out of the vascular compartment. Several cellular enzymes (e.g., collagenase IV, plasminogen activator, cathepsin B, elastase) are thought to be involved in degradation of BM (2, 11). Suppression of human epithin-like serine protease activity therefore can be used to suppress tumor cell invasion and metastasis.

2. Tumor Angiogenesis. Basic fibroblast growth factor (bFGF) has been extracted from the subendothelial extracellular matrix produced in vitro (3) and from basement membranes of the cornea (4), suggesting that extracellular matrix may serve as a reservoir for bFGF. Immunohistochemical staining revealed the localization of bFGF in basement membranes of diverse tissues and blood vessels (5). Despite the ubiquitous presence of bFGF in normal tissues, endothelial cell proliferation in these tissues is usually very low, which suggests that bFGF is somehow sequestered from its site of action. It is possible, therefore, that suppression of human epithin-like serine protease activity can suppress release of active bFGF from extracellular matrix and basement membranes. In addition, displacement of bFGF from its storage within basement membranes and extracellular matrix may therefore provide a novel mechanism for induction of neovascularization in normal and pathological situations. Restriction of endothelial cell growth factors in the extracellular matrix may prevent their systemic action on the vascular endothelium, thus maintaining a very low rate of endothelial cells turnover and vessel growth. On the other hand, release of bFGF from storage in the extracellular matrix may elicit localized endothelial cell proliferation and neovascularization in processes such as wound healing, inflammation and tumor development (6, 7).

3. Inflammation and Cellular Immunity. Epithin-like serine protease activity may be involved in the ability of activated cells of the immune system to leave the circulation and elicit both inflammatory and autoimmune responses. Thus, inflammation and cellular immunity may be regulated by regulating activity of epithin-like serine protease.

4. Viral infection. Removal of the cell surface components by epithin-like serine protease may influence the ability of viruses to attach to the cell surface. Regulation of epithin-like serine protease may therefore be used to treat viral infections.

5. Neurodegenerative diseases. It is also possible that epithin-like serine protease activity can be used to degrade, for example, prion protein amyloid plaques of Genstmann-Straussler Syndrome, Creutzfeldt-Jakob disease, and Scrapie.

6. Restenosis and Atherosclerosis. Proliferation of arterial smooth muscle cells (SMCs) in response to endothelial injury and accumulation of cholesterol rich lipoproteins are basic events in the pathogenesis of atherosclerosis and restenosis (8). It is possible that epithin-like serine protease may be involved in the catabolic pathway that may allow substantial cellular and interstitial accumulation of cholesterol rich lipoproteins. The latter pathway is expected to be highly atherogenic by promoting accumulation of apoB and apoE rich lipoproteins (i.e. LDL, VLDL, chylomicrons), independent of feedback inhibition by the cellular sterol content. Altered levels of human epithin-like serine protease activity therefore may inhibit both SMC proliferation and lipid accumulation and thus may halt the progression of restenosis and atherosclerosis.

7. Chronic obstructive pulmonary (or airways) disease (COPD) is a condition defined physiologically as airflow obstruction that generally results from a mixture of emphysema and peripheral airway obstruction due to chronic bronchitis (Senior & Shapiro, Pulmonary Diseases and Disorders, 3d ed., New York, McGraw-Hill, 1998, pp. 659-681, 1998; Barnes, Chest 117, 10S-14S, 2000). Emphysema is characterized by destruction of alveolar walls leading to abnormal enlargement of the air spaces of the lung. Chronic bronchitis is defined clinically as the presence of chronic productive cough for three months in each of two successive years. In COPD, airflow obstruction is usually progressive and is only partially reversible. By far the most important risk factor for development of COPD is cigarette smoking, although the disease does occur in non-smokers.

Chronic inflammation of the airways is a key pathological feature of COPD (Senior & Shapiro, 1998). The inflammatory cell population comprises increased numbers of macrophages, neutrophils, and CD8⁺ lymphocytes. Inhaled irritants, such as cigarette smoke, activate macrophages which are resident in the respiratory tract, as well as epithelial cells leading to release of chemokines (e.g., interleukin-8) and other chemotactic factors. These chemotactic factors act to increase the neutrophil/monocyte trafficking from the blood into the lung tissue and airways. Neutrophils and monocytes recruited into the airways can release a variety of potentially damaging mediators such as proteolytic enzymes and reactive oxygen species. Matrix degradation and emphysema, along with airway wall thickening, surfactant dysfunction, and mucus hypersecretion, all are potential sequelae of this inflammatory response that lead to impaired airflow and gas exchange.

COPD is characterized by damage to the lung extracellular matrix and emphysema can be viewed as the pathologic process that affects the lung parenchyma. This process eventually leads to the destruction of the airway walls resulting in permanent airspace enlargement (Senior and Shapiro, in PULMONARY DISEASES AND DISORDERS, 3^{rd} ed., New York, McGraw-Hill, 1998, pp. 659-681, 1998). The observation that inherited deficiency of al-antitrypsin (al-AT), the primary inhibitor of neutrophil elastase, predisposes individuals to early onset emphysema, and that intrapulmonary instillation of elastolytic enzymes in experimental animals causes emphysema, led to the elastase:antielastase hypothesis for the pathogenesis of emphysema (Eriksson, Acta Med. Scand. 177(Suppl.), 432, 1965, Gross, J. Occup. Med. 6, 481-84, 1964). This in turn led to the concept that destruction of elastin in the lung parenchyma is the basis of the development of emphysema.

A broad range of immune and inflammatory cells including neutrophils, macrophages, T lymphocytes and eosinophils contain proteolytic enzymes that could contribute to the destruction of lung extracellular matrix (Shapiro, 1999). In addition, a number of different classes of proteases have been identified that have the potential to contribute to lung matrix destruction. These include serine proteases, matrix metalloproteinases and cysteine proteases. Of these classes of enzymes, a number can hydrolyze elastin and have been shown to be elevated in COPD patients (neutrophil elastase, MMP-2, 9, 12) (Culpitt et al., Am. J. Respir. Crit. Care Med. 160, 1635-39, 1999, Shapiro, Am. J. Crit. Care Med. 160 (5), S29-S32,1999).

It is expected that in the future novel members of the existing classes of proteases and new classes of proteases will be identified that play a significant role in the damage of the extracellular lung matrix including elastin proteolysis. Novel protease targets therefore remain very attractive therapeutic targets.

8. Other therapeutic and diagnostic indications. Anti-human epithin-like serine protease antibodies can be applied for immunodetection and diagnosis of micrometastases, autoimmune lesions, and renal failure in biopsy specimens, plasma samples, and body fluids. Alternatively, if desired an epithin-like serine protease function can be supplied to a cell by introducing an epithin-like serine protease-encoding polynucleotide into the cell.

The invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a polypeptide-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects epithin-like serine protease activity can be administered to a human cell, either in vitro or in vivo, to reduce epithin-like serine protease activity. The reagent preferably binds to an expression product of a human epithin-like serine protease gene. If the expression product is a polypeptide, the reagent is preferably an antibody. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung or liver.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmol of liposome delivered to about 10^{≢}cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a tumor cell, such as a tumor cell ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Determination of a Therapeutically Effective Dose

The determination of a therapeutically effective dose is well within the capability of those skilled in the art. A therapeutically effective dose refers to that amount of active ingredient which increases or decreases extracellular matrix degradation relative to that which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of an epithin-like serine protease polynucleotide or activity of an epithin-like serine protease polypeptide by at least about 10, preferably about 50, more preferably about 75, 90, or 100% relative to the absence of the reagent. The effectiveness of the mechanism chosen to decrease the level of expression of an epithin-like serine protease polynucleotide or the activity of an epithin-like serine protease polypeptide can be assessed using methods well known in the art, such as hybridization of nucleotide probes to epithin-like serine protease-specific mRNA, quantitative RT-PCR, immunologic detection of an epithin-like serine protease polypeptide, or measurement of epithin-like serine protease activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

The above disclosure generally describes the present invention, and all patents and patent applications cited in this disclosure are expressly incorporated herein. A more complete understanding can be obtained by reference to the following specific examples which are provided for purposes of illustration only and are not intended to limit the scope of the invention.

### EXAMPLE 1

### Detection of Epithin-Like Serine Protease Activity

The polynucleotide of SEQ ID NO: 26 is inserted into the expression vector pCEV4 and the expression vector pCEV4-epithin-like serine protease polypeptide obtained is transfected into human embryonic kidney 293 cells. From these cells cellular extracts are obtained and protease activity is measured using thiobenzylester substrates, as described in U.S. Pat. No. 5,500,344. For monitoring enzyme activities from granules and column fractions, assays are performed at room temperature using 0.5 mM 5,5′-dithiobis-(2-nitrobenzoic acid) (DTNB) (Sigma) to detect the HSBz1 leaving group (ε₄₁₀=13600 M⁻¹ cm⁻¹).

BLT-esterase activity is estimated using a microtiter assay (Green and Shaw, Anal. Biochem. 93, 223-226, 1979). Briefly, 50 μl of sample is added to 100 μl of 1 mM DTNB, made up in 10 mM HEPES, 1 mM CaCl₂, 1 mM MgCl₂, pH 7.2. The reaction is initiated by the addition of 50 μl of BLT (Sigma) to give a final concentration of 500 μM. For Metase determinations, 50 μl of dilutions of the sample in 0.1 M HEPES, 0.05 M CaCl₂, pH 7.5, are added to 100 μl of 1 mM DTNB, and the reaction is initiated by the addition of 50 μl of Boc-Ala-Ala-Met-S Benzyl (Bzl) to give a final concentration of 150 μM. The duration of the assay depends on color development, the rate of which is measured (O.D.₄₁₀) on a Dynatech MR 5000 microplate reader. Controls of sample and DTNB alone or DTNB and substrate alone are run.

For more sensitive comparisons of enzymatic activities, peptide thiobenzyl ester substrates are used to measure protease activities. The chymase substrate Suc-Phe-Leu-Phe-SBzl is purchased from BACHEM Bioscience Inc., Philadelphia, Pa. Z-Arg-SBzl (the tryptase substrate, Kam et al. J. Biol. Chem. 262, 3444-3451, 1987): Boc-Ala-Ala-AA-SBzl (AA=Asp, Met, Lcu, Nle, or Ser), and Suc-Ala-Ala-Met-SBzl (Odake et al. Biochemistry 30, 2217-2227, 1991); Harper et al., Biochemistry 23, 2995-3002, 1984) are synthesized previously. Boc-Ala-Ala-Asp-SBzl is the substrate for Asp-ase and peptide thiobenzyl esters containing Met, Leu or Nle are substrates for Met-ase SP. Assays are performed at room temperature in 0.1 M, HEPES buffer, pH 7.5, containing 0.01 M CaCl₂ and 8% Me₂O using 0.34 mM 4,4′-dithiodipyridine (Aldrithiol-4, Aldrich Chemical Co., Milwaukee, Wis.) to detect HSBzl leaving group that reacts with 4,4′-dithiodipyridine to release thiopyridone (ε324=19800 M⁻¹ cm⁻¹, Grasetti and Murray, Arch. Biochem. Biophys. 119, 41-49, 1967). The initial rates are measured at 324 nm using a Beckman 35 spectrophotometer when 10-25 μl of an enzyme stock solution is added to a cuvette containing 2.0 ml of buffer, 150 μl of 4,4′-dithiodipyridine, and 25 μl of substrate. The same volume of substrate and 4,4′-dithiodipyridine are added to the reference cell in order to compensate for the background hydrolysis rate of the substrates. Initial rates are measured in duplicate for each substrate concentration and are averaged in each case. Substrate concentrations are 100-133 μM. It is shown that the polypeptide of SEQ ID NO: 27 has an epithin-like serine protease activity.

### EXAMPLE 2

Identification of a Test Compound Which Binds to an Epithin-Like Serine Protease Polypeptide

Purified epithin-like serine protease polypeptides comprising a glutathione-S-transferase protein and absorbed onto glutathione-derivatized wells of 96-well microtiter plates are contacted with test compounds from a small molecule library at pH 7.0 in a physiological buffer solution. Epithin-like serine protease polypeptides comprise an amino acid sequence shown in SEQ ID NO:10, 24 or 27. The test compounds comprise a fluorescent tag. The samples are incubated for 5 minutes to one hour. Control samples are incubated in the absence of a test compound.

The buffer solution containing the test compounds is washed from the wells. Binding of a test compound to an epithin-like serine protease polypeptide is detected by fluorescence measurements of the contents of the wells. A test compound which increases the fluorescence in a well by at least 15% relative to fluorescence of a well in which a test compound was not incubated is identified as a compound which binds to an epithin-like serine protease polypeptide.

### EXAMPLE 3

Identification of a Test Compound Which Decreases Epithin-Like Serine Protease Activity

Cellular extracts from the human colon cancer cell line HCT116 are contacted with test compounds from a small molecule library and assayed for epithin-like serine protease activity. Control extracts, in the absence of a test compound, also are assayed. Protease activity can be measured using thiobenzylester substrates, as described in U.S. Pat. No. 5,500,344. For monitoring enzyme activities from granules and column fractions, assays are performed at room temperature using 0.5 mM 5,5′-dithiobis-(2-nitrobenzoic acid) (DTNB) (Sigma) to detect the HSBzl leaving group (ε₄₁₀=13600 M⁻¹ cm⁻¹).

BLT-esterase activity is estimated using a microtiter assay (Green and Shaw, Anal. Biochem. 93, 223-226, 1979). Briefly, 50 μl of sample is added to 100 μl of 1 mM DTNB, made up in 10 mM HEPES, 1 mM CaCl₂, 1 mM MgCl₂, pH 7.2. The reaction is initiated by the addition of 50 μl of BLT (Sigma) to give a final concentration of 500 μM. For Metase determinations, 50 μl of dilutions of the sample in 0.1 M HEPES, 0.05 M CaCl₂, pH 7.5, are added to 100 μl of 1 mM DTNB, and the reaction is initiated by the addition of 50 μl of Boc-Ala-Ala-Met-S Benzyl (Bzl) to give a final concentration of 150 μM. The duration of the assay depends on color development, the rate of which is measured (O.D.₄₁₀) on a Dynatech MR 5000 microplate reader. Controls of sample and DTNB alone or DTNB and substrate alone are run.

For more sensitive comparisons of enzymatic activities, peptide thiobenzyl ester substrates are used to measure protease activities. The chymase substrate Suc-Phe-Leu-Phe-SBzl is purchased from BACHEM Bioscience Inc., Philadelphia, Pa. Z-Arg-SBzl (the tryptase substrate, Kam et al., J. Biol. Chem. 262, 3444-3451, 1987); Boc-Ala-Ala-AA-SBzl (AA=Asp, Met, Leu, Nle, or Ser), and Suc-Ala-Ala-Met-SBzl (Odake et al, Biochemistry 30, 2217-2227, 1991); Harper et al., Biochemistry 23, 2995-3002, 1984) are synthesized previously. Boc-Ala-Ala-Asp-SBzl is the substrate for Asp-ase and peptide thiobenzyl esters containing Met, Leu or Nle are substrates for Met-ase SP. Assays are performed at room temperature in 0.1 M, HEPES buffer, pH 7.5, containing 0.01 M CaCl₂ and 8% Me₂O using 0.34 mM 4,4′-dithiodipyridine (Aldrithiol-4, Aldrich Chemical Co., Milwaukee, Wis.) to detect HSBzl leaving group that reacts with 4,4′-dithiodipyridine to release thiopyridone (ε324=19800 M⁻¹ cm⁻¹, Grasetti and Murray, Arch. Biochem. Biophys. 119, 41-49, 1967). The initial rates are measured at 324 nm using a Beckman 35 spectrophotometer when 10-25 μl of an enzyme stock solution is added to a cuvette containing 2.0 ml of buffer, 150 μl of 4,4′-dithiodipyridine, and 25 μl of substrate. The same volume of substrate and 4,4′-dithiodipyridine are added to the reference cell in order to compensate for the background hydrolysis rate of the substrates. Initial rates are measured in duplicate for each substrate concentration and are averaged in each case. Substrate concentrations are 100-133 μM.

A test compound which decreases epithin-like serine protease activity of the extract relative to the control extract by at least 20% is identified as an epithin-like serine protease inhibitor.

### EXAMPLE 4

Identification of a Test Compound which Decreases Epithin-Like Serine Protease Gene Expression

A test compound is administered to a culture of the breast tumor cell line MDA-468 and incubated at 37° C. for 10 to 45 minutes. A culture of the same type of cells incubated for the same time without the test compound provides a negative control.

RNA is isolated from the two cultures as described in Chirgwin et al., Biochem. 18, 5294-99, 1979). Northern blots are prepared using 20 to 30 μg total RNA and hybridized with a ³²P-labeled epithin-like serine protease-specific probe at 65° C. in Express-hyb (CLONTECH). The probe comprises at least 11 contiguous nucleotides selected from the complement of SEQ ID NO:25 or 26. A test compound which decreases the epithin-like serine protease-specific signal relative to the signal obtained in the absence of the test compound is identified as an inhibitor of epithin-like serine protease gene expression.

### EXAMPLE 5

Treatment of a Breast Tumor with a Reagent which Specifically Binds to an Epithin-Like Serine Protease Gene Product

Synthesis of antisense epithin-like serine protease oligonucleotides comprising at least 11 contiguous nucleotides selected from the complement of SEQ ID NO: 25 or 26 is performed on a Pharmacia Gene Assembler series synthesizer using the phosphoramidite procedure (Uhlmann et al., Chem. Rev. 90, 534-83, 1990). Following assembly and deprotection, oligonucleotides are ethanol-precipitated twice, dried, and suspended in phosphate-buffered saline (PBS) at the desired concentration. Purity of these oligonucleotides is tested by capillary gel electrophoreses and ion exchange HPLC. Endotoxin levels in the oligonucleotide preparation are determined using the Limulus Amebocyte Assay (Bang, Biol. Bull. (Woods Hole, Mass.) 105, 361-362, 1953).

An aqueous composition containing the antisense oligonucleotides at a concentration of 0.1-100 μM is injected directly into a breast tumor with a needle. The needle is placed in the tumors and withdrawn while expressing the aqueous composition within the tumor.

The breast tumor is monitored over a period of days or weeks. Additional injections of the antisense oligonucleotides can be given during that time. Metastasis of the breast tumor is suppressed due to decreased epithin-like serine protease activity of the breast tumor cells.

### EXAMPLE 6

Expression of Recombinant Human Epithin-Like Serine Protease

The Pichia pastoris expression vector pPICZB (Invitrogen, San Diego, Calif.) is used to produce large quantities of recombinant human epithin-like serine protease polypeptides in yeast. The epithin-like serine protease-encoding DNA sequence is derived from SEQ ID NO: 10, 24 or 27. Before insertion into vector pPICZB, the DNA sequence is modified by well known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZ B with the corresponding restriction enzymes the modified DNA sequence is ligated into pPICZB. This expression vector is designed for inducible expression in Pichia pastoris, driven by a yeast promoter. The resulting pPICZ/md-His6 vector is used to transform the yeast.

The yeast is cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the polypeptide from the His6 reporter tag is accomplished by site-specific proteolysis using enterokinase (Invitrogen, San Diego, Calif.) according to manufacturer's instructions. Purified human epithin-like serine protease polypeptide is obtained.

### EXAMPLE 7

Proliferation Inhibition Assay: Antisense Oligonucleotides Suppress the Growth of Cancer Cell Lines

The cell line used for testing is the human colon cancer cell line HCT116. Cells are cultured in RPMI-1640 with 10-15% fetal calf serum at a concentration of 10,000 cells per milliliter in a volume of 0.5 ml and kept at 37° C. in a 95% air/5% CO₂ atmosphere.

Phosphorothioate oligoribonucleotides are synthesized on an Applied Biosystems Model 380B DNA synthesizer using phosphoroamidite chemistry. A sequence of 24 bases complementary to the nucleotides at position 1 to 24 of SEQ ID NO:25 or 26 is used as the test oligonucleotide. As a control, another (random) sequence is used: 5′-TCA ACT GAC TAG ATG TAC ATG GAC-3′. Following assembly and deprotection, oligonucleotides are ethanol-precipitated twice, dried, and suspended in phosphate buffered saline at the desired concentration. Purity of the oligonucleotides is tested by capillary gel electrophoresis and ion exchange HPLC. The purified oligonucleotides are added to the culture medium at a concentration of 10 μM once per day for seven days.

The addition of the test oligonucleotide for seven days results in significantly reduced expression of human epithin-like serine protease as determined by Western blotting. This effect is not observed with the control oligonucleotide. After 3 to 7 days, the number of cells in the cultures is counted using an automatic cell counter. The number of cells in cultures treated with the test oligonucleotide (expressed as 100%) is compared with the number of cells in cultures treated with the control oligonucleotide. The number of cells in cultures treated with the test oligonucleotide is not more than 30% of control, indicating that the inhibition of human epithin-like serine protease has an anti-proliferative effect on cancer cells.

### EXAMPLE 8

In vivo Testing of Compounds/Target Validation

1. Acute Mechanistic Assays

1.1. Reduction in Mitogenic Plasma Hormone Levels

This non-tumor assay measures the ability of a compound to reduce either the endogenous level of a circulating hormone or the level of hormone produced in response to a biologic stimulus. Rodents are administered test compound (p.o., i.p., i.v., i.m., or s.c.). At a predetermined time after administration of test compound, blood plasma is collected. Plasma is assayed for levels of the hormone of interest. If the normal circulating levels of the hormone are too low and/or variable to provide consistent results, the level of the hormone may be elevated by a pre-treatment with a biologic stimulus (i.e., LHRH may be injected i.m. into mice at a dosage of 30 ng/mouse to induce a burst of testosterone synthesis). The timing of plasma collection would be adjusted to coincide with the peak of the induced hormone response. Compound effects are compared to a vehicle-treated control group. An F-test is preformed to determine if the variance is equal or unequal followed by a Student's t-test. Significance is p value≦0.05 compared to the vehicle control group.

1.2. Hollow Fiber Mechanism of Action Assay

Hollow fibers are prepared with desired cell line(s) and implanted intraperitoneally and/or subcutaneously in rodents. Compounds are administered p.o., i.p., i.v., i.m., or s.c. Fibers are harvested in accordance with specific readout assay protocol, these may include assays for gene expression (bDNA, PCR, or Taqman), or a specific biochemical activity (i.e., cAMP levels. Results are analyzed by Student's t-test or Rank Sum test after the variance between groups is compared by an F-test, with significance at p≦0.05 as compared to the vehicle control group.

2. Subacute Functional In Vivo Assays

2.1. Reduction in Mass of Hormone Dependent Tissues

This is another non-tumor assay that measures the ability of a compound to reduce the mass of a hormone dependent tissue (i.e., seminal vesicles in males and uteri in females). Rodents are administered test compound (p.o., i.p., i.v., i.m., or s.c.) according to a predetermined schedule and for a predetermined duration (i.e., 1 week). At termination of the study, animals are weighed, the target organ is excised, any fluid is expressed, and the weight of the organ is recorded. Blood plasma may also be collected. Plasma may be assayed for levels of a hormone of interest or for levels of test agent. Organ weights may be directly compared or they may be normalized for the body weight of the animal. Compound effects are compared to a vehicle-treated control group. An F-test is preformed to determine if the variance is equal or unequal followed by a Student's t-test. Significance is p value≦0.05 compared to the vehicle control group.

2.2. Hollow Fiber Proliferation Assay

Hollow fibers are prepared with desired cell line(s) and implanted intraperitoneally and/or subcutaneously in rodents. Compounds are administered p.o., i.p. i.v., i.m., or s.c. Fibers are harvested in accordance with specific readout assay protocol. Cell proliferation is determined by measuring a marker of cell number (i.e., MTT or LDH). The cell number and change in cell number from the starting inoculum are analyzed by Student's t-test or Rank Sum test after the variance between groups is compared by an F-test, with significance at p≦0.05 as compared to the vehicle control group.

2.3. Anti-Angiogenesis Models

2.3.1. Corneal Angiogenesis

Hydron pellets with or without growth factors or cells are implanted into a micropocket surgically created in the rodent cornea. Compound administration may be systemic or local (compound mixed with growth factors in the hydron pellet). Corneas are harvested at 7 days post implantation immediately following intracardiac infusion of colloidal carbon and are fixed in 10% formalin. Readout is qualitative scoring and/or image analysis. Qualitative scores are compared by Rank Sum test. Image analysis data is evaluated by measuring the area of neovascularization (in pixels) and group averages are compared by Student's t-test (2 tail). Significance is p≦0.05 as compared to the growth factor or cells only group.

2.3.2. Matrigel Angiogenesis

Matrigel, containing cells or growth factors, is injected subcutaneously. Compounds are administered p.o., i.p., i.v., i.m., or s.c. Matrigel plugs are harvested at predetermined time point(s) and prepared for readout. Readout is an ELISA-based assay) for hemoglobin concentration and/or histological examination (i.e. vessel count special staining for endothelial surface markers: CD31, factor-8). Readouts are analyzed by Student's t-test, after the variance between groups is compared by an F-test, with significance determined at p≦0.05 as compared to the vehicle control group.

3. Primary Antitumor Efficacy

3.1. Early Therapy Models

3.1.1. Subcutaneous Tumor

Tumor cells or fragments are implanted subcutaneously on Day 0. Vehicle and/or compounds are administered p.o., i.p., i.v., i.m., or s.c. according to a predetermined schedule starting at a time, usually on Day 1, prior to the ability to measure the tumor burden. Body weights and tumor measurements are recorded 2-3 times weekly. Mean net body and tumor weights are calculated for each data collection day. Anti-tumor efficacy may be initially determined by comparing the size of treated (T) and control (C) tumors on a given day by a Student's t-test, after the variance between groups is compared by an F-test, with significance determined at p≦0.05. The experiment may also be continued past the end of dosing in which case tumor measurements would continue to be recorded to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p≦0.05.

3.1.2. Intraperitoneal/Intracranial Tumor Models

Tumor cells are injected intraperitoneally or intracranially on Day 0. Compounds are administered p.o., i.p., i.v., i.m., or s.c. according to a predetermined schedule starting on Day 1. Observations of morbidity and/or mortality are recorded twice daily. Body weights are measured and recorded twice weekly. Morbidity/mortality data is expressed in terms of the median time of survival and the number of long-term survivors is indicated separately. Survival times are used to generate Kaplan-Meier curves. Significance is p≦0.05 by a log-rank test compared to the control group in the experiment.

3.2. Established Disease Model

Tumor cells or fragments are implanted subcutaneously and grown to the desired size for treatment to begin. Once at the predetermined size range, mice are randomized into treatment groups. Compounds are administered p.o., i.p., i.v., i.m., or s.c. according to a predetermined schedule. Tumor and body weights are measured and recorded 2-3 times weekly. Mean tumor weights of all groups over days post inoculation are graphed for comparison. An F-test is preformed to determine if the variance is equal or unequal followed by a Student's t-test to compare tumor sizes in the treated and control groups at the end of treatment. Significance is p≦0.05 as compared to the control group. Tumor measurements may be recorded after dosing has stopped to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p value≦0.05 compared to the vehicle control group.

3.3. Orthotopic Disease Models

3.3.1. Mammary Fat Pad Assay

Tumor cells or fragments, of mammary adenocarcinoma origin, are implanted directly into a surgically exposed and reflected mammary fat pad in rodents. The fat pad is placed back in its original position and the surgical site is closed. Hormones may also be administered to the rodents to support the growth of the tumors. Compounds are administered p.o., i.p., i.v., i.m., or s.c. according to a predetermined schedule. Tumor and body weights are measured and recorded 2-3 times weekly. Mean tumor weights of all groups over days post inoculation are graphed for comparison. An F-test is preformed to determine if the variance is equal or unequal followed by a Student's t-test to compare tumor sizes in the treated and control groups at the end of treatment. Significance is p≦0.05 as compared to the control group.

Tumor measurements may be recorded after dosing has stopped to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p value≦0.05 compared to the vehicle control group. In addition, this model provides an opportunity to increase the rate of spontaneous metastasis of this type of tumor. Metastasis can be assessed at termination of the study by counting the number of visible foci per target organ, or measuring the target organ weight. The means of these endpoints are compared by Student's t-test after conducting an F-test, with significance determined at p≦0.05 compared to the control group in the experiment.

3.3.2. Intraprostatic Assay

Tumor cells or fragments, of prostatic adenocarcinoma origin, are implanted directly into a surgically exposed dorsal lobe of the prostate in rodents. The prostate is externalized through an abdominal incision so that the tumor can be implanted specifically in the dorsal lobe while verifying that the implant does not enter the seminal vesicles. The successfully inoculated prostate is replaced in the abdomen and the incisions throught e abdomen and skin are closed. Hormones may also be administered to the rodents to support the growth of the tumors. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (i.e., the lungs), or measuring the target organ weight (i.e., the regional lymph nodes). The means of these endpoints are compared by Student's t-test after conducting an F-test, with significance determined at p≦0.05 compared to the control group in the experiment.

3.3.3. Intrabronchial Assay

Tumor cells of pulmonary origin may be implanted intra-bronchially by making an incision through the skin and exposing the trachea. The trachea is pierced with the beveled end of a 25 gauge needle and the tumor cells are inoculated into the main bronchus using a flat-ended 27 gauge needle with a 90° bend. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (i.e., the contralateral lung), or measuring the target organ weight. The means of these endpoints are compared by Student's t-test after conducting an F-test, with significance determined at p≦0.05 compared to the control group in the experiment.

3.3.4. Intracecal Assay

Tumor cells of gastrointestinal origin may be implanted intracecally by making an abdominal incision through the skin and externalizing the intestine. Tumor cells are inoculated into the cecal wall without penetrating the lumen of the intestine using a 27 or 30 gauge needle. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (i.e., the liver), or measuring the target organ weight. The means of these endpoints are compared by Student's t-test after conducting an F-test, with significance determined at p≦0.05 compared to the control group in the experiment.

4. Secondary (Metastatic) Antitumor Efficacy

4.1. Spontaneous Melastasis

Tumor cells are inoculated s.c. and the tumors allowed to grow to a predetermined range for spontaneous metastasis studies to the lung or liver. These primary tumors are then excised. Compounds are administered p.o., i.p., i.v., i.m., or s.c. according to a predetermined schedule which may include the period leading up to the excision of the primary tumor to evaluate therapies directed at inhibiting the early stages of tumor metastasis. Observations of morbidity and/or mortality are recorded daily. Body weights are measured and recorded twice weekly. Potential endpoints include survival time, numbers of visible foci per target organ, or target organ weight. When survival time is used as the endpoint the other values are not determined. Survival data is used to generate Kaplan-Meier curves. Significance is p≦0.05 by a log-rank test compared to the control group in the experiment. The mean number of visible tumor foci, as determined under a dissecting microscope, and the mean target organ weights are compared by Student's t-test after conducting an F-test, with significance determined at p≦0.05 compared to the control group in the experiment for both of these endpoints.

4.2. Forced Metastasis

Tumor cells are injected into the tail vein, portal vein, or the left ventricle of the heart in experimental (forced) lung, liver, and bone metastasis studies, respectively. Compounds are administered p.o., i.p., i.v., i.m., or s.c. according to a predetermined schedule. Observations of morbidity and/or mortality are recorded daily. Body weights are measured and recorded twice weekly. Potential endpoints include survival time, numbers of visible foci per target organ, or target organ weight. When survival time is used as the endpoint the other values are not determined. Survival data is used to generate Kaplan-Meier curves. Significance is p≦0.05 by a log-rank test compared to the control group in the experiment. The mean number of visible tumor foci, as determined under a dissecting microscope and the mean target organ weights are compared by Student's t-test after conducting an F-test, with significance at p≦0.05 compared to the vehicle control group in the experiment for both endpoints.

### EXAMPLE 9

Tissue-Specific Expression of Epithin-Like Serine Protease

As a first step to establishing a role for epithin-like serine protease in the pathogenesis of COPD, expression profiling of the gene was done using real-time quantitative PCR (TaqMan) with RNA samples isolated from a wide range of human cells and tissues. Total RNA samples were either purchased from commercial suppliers or purified in-house. Two panels of RNAs were used for profiling: a whole body organ panel (Table 1) and a respiratory specific panel (Table 2).

Real-time quantitative PCR. This technique is a development of the kinetic analysis of PCR first described by Higuchi et al. (BioTechnology 10, 413-17, 1992; BioTechnology 11, 1026-30, 1993). The principle is that at any given cycle within the exponential phase of PCR, the amount of product is proportional to the initial number of template copies. PCR amplification is performed in the presence of an oligonucleotide probe (TaqMan probe) that is complementary to the target sequence and labeled with a fluorescent reporter dye and a quencher dye. During the extension phase of PCR, the probe is cleaved by the 5′-3′ endonuclease activity of Taq DNA polymerase, releasing the fluorophore from the effect of the quenching dye (Holland et al., Proc. Natl. Acad. Sci. U.S.A. 88, 7276-80, 1991). Because the fluorescence emission increases in direct proportion to the amount of the specific amplified product, the exponential growth phase of PCR product can be detected and used to determine the initial template concentration (Heid et al., Genome Res. 6, 986-94, 1996, and Gibson et al., Genoine Res. 6, 995-1001, 1996).

RNA extraction and cDNA preparation. Total RNA from each of the ‘in-house’ samples listed in Table 2 was isolated using Qiagen's (Crawley, West Sussex, UK) RNeasy system according to the manufacturer's protocol. The concentration of purified RNA was determined using RiboGreen RNA quantitation kit (Molecular Probes Europe, The Netherlands). RNA concentrations of the samples purchased from commercial suppliers were also determined using RiboGreen. For the preparation of cDNA, 1 μg of total RNA was reverse transcribed using 200U of SUPERSCRIPT™ II RNaseH⁻Reverse Transcriptase (Life Technologies, Paisley, UK), 10 mM dithiothreitol, 0.5 mM of each dNTP, and 5 μM random hexamers (PE Applied Biosystems, Warrington, Cheshire, UK) in a final volume of 20 μl according to the manufacturer's protocol.

TaqMan quantitative analysis. Specific primers and probe were designed according to the recommendations of PE Applied Biosystems and are listed below:

| | |
|---|---|
| Forward primer: | 5′-CTTCCTAGGCACGCCGTTC-3′ |
| Reverse primer: | 5′-AGCAGCGCCACGTCGTAG-3′ |
| Probe: | 5′-(FAM)-CGCATCTACAAGCACCCGTTCTACA |
| | ATCTCT-3′ |

where FAM=6-carboxy-fluorescein.

Quantitative PCR was performed with long of reverse transcribed RNA from each sample. Each determination was done in duplicate.

The assay reaction mix was as follows: 1× final TaqMan Universal PCR Master Mix (from 2× stock) (PE Applied Biosystems, CA); 900 nM forward primer; 90 nM reverse primer; 200 nM probe; 10 ng cDNA; and water to 25 μl.

Each of the following steps were carried out once: pre PCR, 2 minutes at 50° C., and 10 minutes at 95° C. The following steps are carried out 40 times: denaturation, 15 seconds at 95° C., annealing/extension, 1 minute at 60° C.

Real-time quantitative PCR was done using an ABI Prism 7700 Sequence Detector. The C_{T} value generated for each reaction was used to determine the initial template concentration (copy number) by interpolation from a universal standard curve. The level of expression of the target gene in each sample was calculated relative to the sample with the lowest expression of the gene.

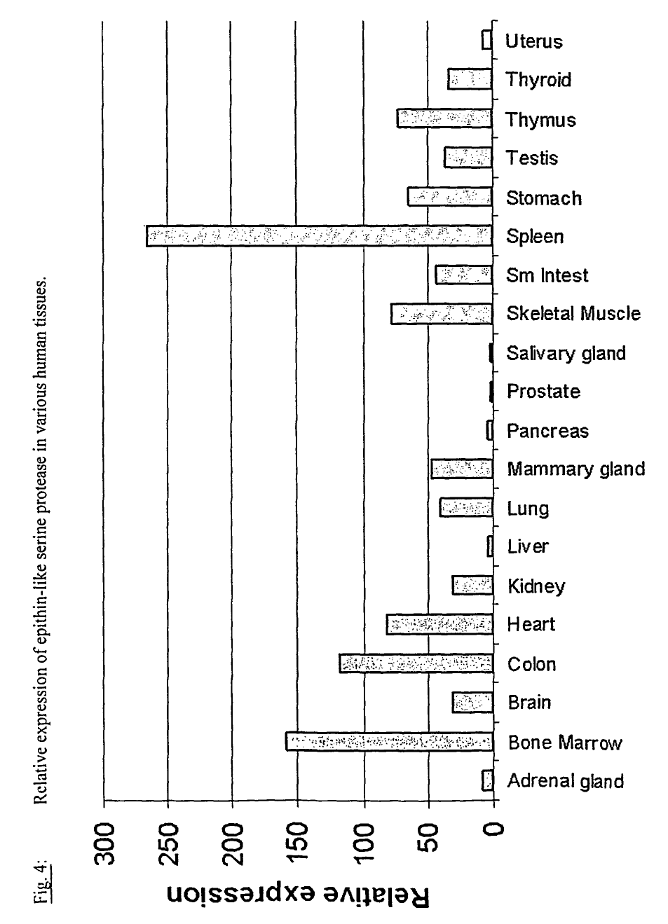

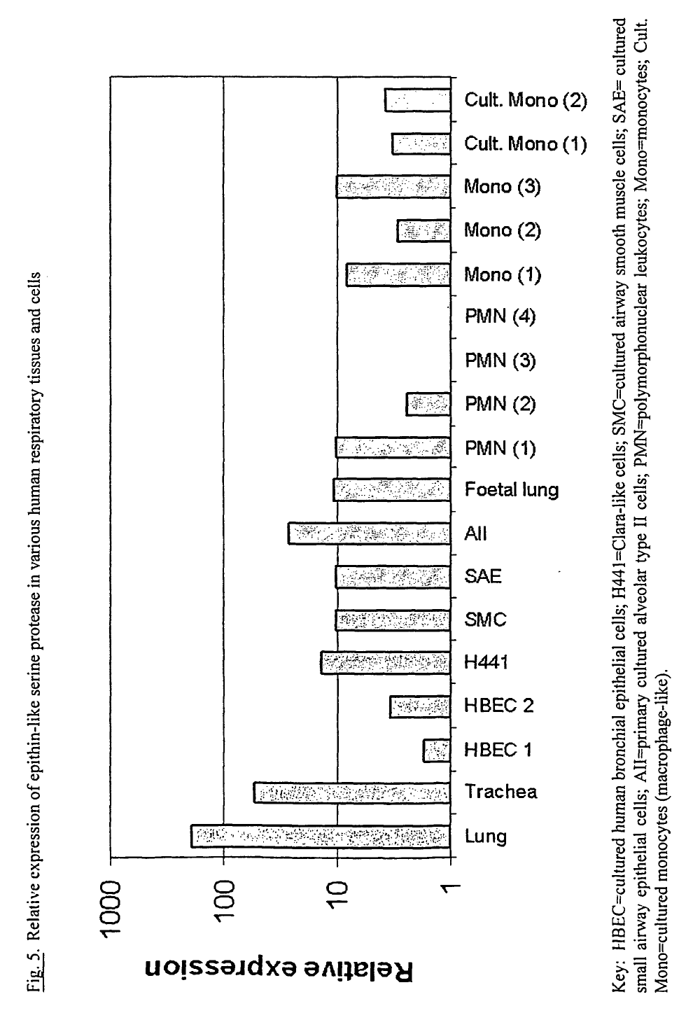

The relative expression of epithin-like serine protease across various human tissues is shown in FIG. 4. Expression of the gene was detected in all tissues tested and was especially abundant in spleen and bone marrow. Of particular interest was the expression of epithin-like serine protease in lung and this was investigated further by analyzing the expression of the gene in some of the constituent cell types of the lung. Again, expression was detected in all of the tissue and cell types tested, albeit that expression was not found in two out of four samples from polymorphonuclear leukocytes (FIG. 5). All of the cell types tested had a much lower level of expression of the protease than that found in whole lung, suggesting that the most important site(s) of expression of the gene is not represented in the RNA panel used in these studies.

Epithin-like serine protease is an apparent human ortholog of mouse epithin, a type II membrane serine protease (Kim, et al. Immunogenetics 49, 420-428, 1999). Although the function of epithin is not known, it is likely involved in the processing of peptides or proteins at cell surfaces. In the lung it is possible that epithin-like serine protease is involved in tissue remodeling, and that dysfunction or dysregulation of the protease could play a significant role in the destruction of the lung matrix in diseases such as COPD. Epithin-like serine protease, therefore, represents a potential therapeutic target for COPD.

TABLE 1
Human organ RNA panel used for real-time quantitative PCR.
All samples were obtained from Clontech UK Ltd. Basingstoke, UK.

| Tissue | Cat. # |
|---|---|
| Adrenal gland | Human Panel V, K4004-1 |
| Bone marrow | Human Panel II, K4001-1 |
| Brain | Human Panel I, K4000-1 |
| Colon | Human Panel II, K4001-1 |
| Heart | Human Panel III, K4002-1 |
| Kidney | Human Panel I, K4000-1 |
| Liver | Human Panel I, K4000-1 |
| Lung | Human Panel I, K4000-1 |
| Mammary gland | Human Panel III, K4002-1 |
| Pancreas | Human Panel V, K4004-1 |
| Prostate | Human Panel III, K4002-1 |
| Salivary gland | Human Panel V, K4004-1 |
| Skeletal muscle | Human Panel III, K4002-1 |
| Small intestine | Human Panel II, K4001-1 |
| Spleen | Human Panel II, K4001-1 |
| Stomach | Human Panel II, K4001-1 |
| Testis | Human Panel III, K4002-1 |
| Thymus | Human Panel II, K4001-1 |
| Thyroid | Human Panel V, K4004-1 |
| Uterus | Human Panel III, K4002-1 |

TABLE 2
Human respiratory specific RNA panel used for real-time
quantitative PCR.

| Tissue/cell type | Supplier, cat # |
|---|---|
| Lung (fetal) | Takara (Japan) |
| Lung | Clontech, Human Panel I, |
| | K4000-1 |
| Trachea | Clontech, Human Panel I, |
| | K4000-1 |
| Cultured human bronchial epithelial cells | In-house |
| Cultured airway smooth muscle cells | In-house |
| Cultured small airway epithelial cells | In-house |
| Primary cultured alveolar type II cells | In-house |
| Cultured H441 cells (Clara-like) | In-house |
| Freshly isolated polymorphonuclear | In-house |
| leukocytes (neutrophils) | |
| Freshly isolated monocytes | In-house |
| Cultured monocytes (macrophage-like) | In-house |

### REFERENCES

1. Nicolson (1988) Organ specificity of tumor metastasis: Role of preferential adhesion, invasion and growth of malignant cells at specific secondary sites. Cancer Met. Rev. 7, 143-188.

2. Liotta et al. (1983) Tumor invasion and the extracellular matrix. Lab. Invest. 49, 639-649.

3. Vlodavsky et al. (1987) Endothelial cell-derived basic fibroblast growth factor: Synthesis and deposition into subendothelial extracellular matrix. Proc. Natl. Acad Sci. USA 84, 2292-2296.

4. Folkman et al. (1980) A heparin-binding angiogenic protein—basic fibroblast growth factor—is stored within basement membrane. Am. J. Pathol. 130, 393400.

5. Cardon-Cardo et al. (1990) Expression of basic fibroblast growth factor in normal human tissues. Lab. Invest. 63, 832-840.

6. Vlodavsky et al. (1991) Extracellular sequestration and release of fibroblast growth factor: a regulatory mechanism? Trends Biochem. Sci. 16, 268-271.

7. Vlodavsky et al. (1993) Extracellular matrix-bound growth factors, enzymes and plasma proteins. In BASEMENT MEMBRANES: CELLULAR AND MOLECULAR ASPECTS Rohrbach & Timpl, eds., pp327-343. Academic Press Inc. Orlando, Fla.

8. Ross (1993) The pathogenesis of atherosclerosis: a perspective for the 1990s Nature (Lond.). 362, 801-809.

9. McCarthy et al. (1986). Human fibronectin contains distinct adhesion—and motility-promoting domains for metastatic melanoma cells. J. Cell Biol. 102, 179-88.

10. Van Muijen et al. (1995) Properties of metastasizing and non-metastasizing human melanoma cells. Recent Results in Cancer Research 139, 104-22.

11. Price et al. (1997) The Biochemistry of Cancer Dissemination, in Critical Reviews in Biochemistry and Mol. Biol. 32, 175-253.

1. An isolated polynucleotide encoding a epithin-like serine protease polypeptide and being selected from the group consisting of:
a) a polynucleotide encoding a epithin-like serine protease polypeptide comprising an amino acid sequence selected form the group consisting of:
amino acid sequences which are at least about 50% identical to the amino acid sequence shown in SEQ ID NO: 10;
the amino acid sequence shown in SEQ ID NO: 10;
amino acid sequences which are at least about 50% identical to the amino acid sequence shown in SEQ ID NO: 24;
the amino acid sequence shown in SEQ ID NO: 24;
amino acid sequences which are at least about 50% identical to the amino acid sequence shown in SEQ ID NO: 27; and
the amino acid sequence shown in SEQ ID NO: 27.
b) a polynucleotide comprising the sequence of SEQ ID NO: 25 or 26;
c) a polynucleotide which hybridizes under stringent conditions to a polynucleotide specified in (a) and (b);
d) a polynucleotide the sequence of which deviates from the polynucleotide sequences specified in (a) to (c) due to the degeneration of the genetic code; and
e) a polynucleotide which represents a fragment, derivative or allelic variation of a polynucleotide sequence specified in (a) to (d).

2. An expression vector containing any polynucleotide of claim 1.

3. A host cell containing the expression vector of claim 2.

4. A substantially purified epithin-like serine protease polypeptide encoded by a polynucleotide of claim 1.

5. A method for producing a epithin-like serine protease polypeptide, wherein the method comprises the following steps:
a) culturing the host cell of claim 3 under conditions suitable for the expression of the epithin-like serine protease polypeptide; and
b) recovering the epithin-like serine protease polypeptide from the host cell culture.

6. A method for detection of a polynucleotide encoding a epithin-like serine protease polypeptide in a biological sample comprising the following steps:
a) hybridizing any polynucleotide of claim 1 to a nucleic acid material of a biological sample, thereby forming a hybridization complex; and
b) detecting said hybridization complex.

7. The method of claim 6, wherein before hybridization, the nucleic acid material of the biological sample is amplified.

8. A method for the detection of a polynucleotide of claim 1 or a epithin-like serine protease polypeptide of claim 4 comprising the steps of:
contacting a biological sample with a reagent which specifically interacts with the polynucleotide or the epithin-like serine protease polypeptide.

9. A diagnostic kit for conducting the method of any one of claims 6 to 8.

10. A method of screening for agents which decrease the activity of a epithin-like serine protease, comprising the steps of:
contacting a test compound with any epithin-like serine protease polypeptide encoded by any polynucleotide of claim 1;
detecting binding of the test compound to the epithin-like serine protease polypeptide, wherein a test compound which binds to the polypeptide is identified as a potential therapeutic agent for decreasing the activity of a epithin-like serine protease.

11. A method of screening for agents which regulate the activity of a epithin-like serine protease, comprising the steps of:
contacting a test compound with a epithin-like serine protease polypeptide encoded by any polynucleotide of claim 1; and
detecting a epithin-like serine protease activity of the polypeptide, wherein a test compound which increases the epithin-like serine protease activity is identified as a potential therapeutic agent for increasing the activity of the epithin-like serine protease, and wherein a test compound which decreases the epithin-like serine protease activity of the polypeptide is identified as a potential therapeutic agent for decreasing the activity of the epithin-like serine protease.

12. A method of screening for agents which decrease the activity of a epithin-like serine protease, comprising the steps of:
contacting a test compound with any polynucleotide of claim 1 and detecting binding of the test compound to the polynucleotide, wherein a test compound which binds to the polynucleotide is identified as a potential therapeutic agent for decreasing the activity of epithin-like serine protease.

13. A method of reducing the activity of epithin-like serine protease, comprising the steps of:
contacting a cell with a reagent which specifically binds to any polynucleotide of claim 1 or any epithin-like serine protease polypeptide of claim 4, whereby the activity of epithin-like serine protease is reduced.

14. A reagent that modulates the activity of a epithin-like serine protease polypeptide or a polynucleotide wherein said reagent is identified by the method of any of the claim 10 to 12.

15. A pharmaceutical composition, comprising:
the expression vector of claim 2 or the reagent of claim 14 and a pharmaceutically acceptable carrier.

16. Use of the pharmaceutical composition of claim 15 for modulating the activity of a epithin-like serine protease in a disease.

17. Use of claim 16 wherein the disease is metastasis of malignant cells, tumor angiogenesis, inflammation, atherosclerosis, a neurodegenerative disease, COPD or a pathogenic infection.

18. A cDNA encoding a polypeptide comprising the amino acid sequence shown in SEQ ID NO: 10, 24 and 27.

19. The cDNA of claim 18 which comprises SEQ ID NO: 25 or 26.

20. The cDNA of claim 18 which consists of SEQ ID NO: 25 or 26.

21. An expression vector comprising a polynucleotide which encodes a polypeptide comprising the amino acid sequence shown in SEQ ID NO: 10, 24 and 27.

22. The expression vector of claim 21 wherein the polynucleotide consists of SEQ ID NO: 25 or 26.

23. A host cell comprising an expression vector which encodes a polypeptide comprising the amino acid sequence shown in SEQ ID NO: 10, 24 and 27.

24. The host cell of claim 23 wherein the polynucleotide consists of SEQ ID NO: 25 or 26.

25. A purified polypeptide comprising the amino acid sequence shown in SEQ ID NO: 10, 24 and 27.

26. The purified polypeptide of claim 25 which consists of the amino acid sequence shown in SEQ ID NO10, 24 and 27.

27. A fusion protein comprising a polypeptide having the amino acid sequence shown in SEQ ID NO: 10, 24 and 27.

28. A method of producing a polypeptide comprising the amino acid sequence shown in SEQ ID NO: 10, 24 and 27, comprising the steps of:
culturing a host cell comprising an expression vector which encodes the polypeptide under conditions whereby the polypeptide is expressed; and isolating the polypeptide.

29. The method of claim 28 wherein the expression vector comprises SEQ ID NO: 25 or 26.

30. A method of detecting a coding sequence for a polypeptide comprising the amino acid sequence shown in SEQ ID NO: 10, 12, 13, 14, 15, 16, 17, 18, 19, 20, 21,22, 23, 24 and 27, comprising the steps of:
hybridizing a polynucleotide comprising 11 contiguous nucleotides of SEQ ID NO: 1, 2, 3, 4, 5, 6, 7, 8, 9, 25 or 26 to nucleic acid material of a biological sample, thereby forming a hybridization complex; and
detecting the hybridization complex.

31. The method of claim 30 further comprising the step of amplifying the nucleic acid material before the step of hybridizing.

32. A kit for detecting a coding sequence for a polypeptide comprising the amino acid sequence shown in SEQ ID NO: 10, 12, 13, 14, 15, 16, 17, 18, 19, 20, 21, 22,23, 24 and 27, comprising:
a polynucleotide comprising 11 contiguous nucleotides of SEQ ID NO: 1, 2, 3, 4, 5, 6, 7, 8, 9, 25or 26; and
instructions for the method of claim 30.

33. A method of detecting a polypeptide comprising the amino acid sequence shown in SEQ ID NO: 10, 12, 13, 14, 15, 16, 17, 18, 19, 20, 21, 22, 23, 24 and 27, comprising the steps of:
contacting a biological sample with a reagent that specifically binds to the polypeptide to form a reagent-polypeptide complex; and
detecting the reagent-polypeptide complex.

34. The method of claim 33 wherein the reagent is an antibody.

35. A kit for detecting a polypeptide comprising the amino acid sequence shown in SEQ ID NO: 10, 12, 13, 14, 15, 16, 17, 18, 19, 20, 21, 22, 23, 24 and 27, comprising:
an antibody which specifically binds to the polypeptide; and
instructions for the method of claim 33.

36. A method of screening for agents which can modulate the activity of a human epithin-like serine protease, comprising the steps of:
contacting a test compound with a polypeptide comprising an amino acid sequence selected from the group consisting of: (1) amino acid sequences which are at least about 50% identical to the amino acid sequence shown in SEQ ID NO: 10, 12, 13, 14, 15, 16, 17, 18, 19, 20, 21, 22, 23, 24 and 27 and (2) the amino acid sequence shown in SEQ ID NO: 10, 12, 13, 14, 15, 16, 17, 18, 19, 20, 21, 22, 23, 24 and 27; and
detecting binding of the test compound to the polypeptide, wherein a test compound which binds to the polypeptide is identified as a potential agent for regulating activity of the human epithin-like serine protease.

37. The method of claim 36 wherein the step of contacting is in a cell.

38. The method of claim 36 wherein the cell is in vitro.

39. The method of claim 36 wherein the step of contacting is in a cell-free system.

40. The method of claim 36 wherein the polypeptide comprises a detectable label.

41. The method of claim 36 wherein the test compound comprises a detectable label.

42. The method of claim 36 wherein the test compound displaces a labeled ligand which is bound to the polypeptide.

43. The method of claim 36 wherein the polypeptide is bound to a solid support.

44. The method of claim 36 wherein the test compound is bound to a solid support.

45. A method of screening for agents which modulate an activity of a human epithin-like serine protease, comprising the steps of:
contacting a test compound with a polypeptide comprising an amino acid sequence selected from the group consisting of: (1) amino acid sequences which are at least about 50% identical to the amino acid sequence shown in SEQ ID NO: 10, 12, 13, 14, 15, 16, 17, 18, 19, 20, 21, 22, 23, 24 and 27 and (2) the amino acid sequence shown in SEQ ID NO: 10, 12, 13, 14, 15, 16, 17, 18, 19, 20, 21, 22, 23, 24 and 27; and
detecting an activity of the polypeptide, wherein a test compound which increases the activity of the polypeptide is identified as a potential agent for increasing the activity of the human epithin-like serine protease, and wherein
a test compound which decreases the activity of the polypeptide is identified as a potential agent for decreasing the activity of the human epithin-like serine protease.

46. The method of claim 45 wherein the step of contacting is in a cell.

47. The method of claim 45 wherein the cell is in vitro.

48. The method of claim 45 wherein the step of contacting is in a cell-free system.

49. A method of screening for agents which modulate an activity of a human epithin-like serine protease, comprising the steps of:
contacting a test compound with a product encoded by a polynucleotide which comprises the nucleotide sequence shown in SEQ ID NO: 1, 2, 3, 4, 5, 6, 7, 8, 9, 25 or 26; and
detecting binding of the test compound to the product, wherein a test compound which binds to the product is identified as a potential agent for regulating the activity of the human epithin-like serine protease.

50. The method of claim 49 wherein the product is a polypeptide.

51. The method of claim 49 wherein the product is RNA.

52. A method of reducing activity of a human epithin-like serine protease, comprising the step of:
contacting a cell with a reagent which specifically binds to a product encoded by a polynucleotide comprising the nucleotide sequence shown in SEQ ID NO: 1, 2, 3, 4, 5, 6, 7, 8, 9, 25 or 26, whereby the activity of a human epithin-like serine protease is reduced.

53. The method of claim 52 wherein the product is a polypeptide.

54. The method of claim 53 wherein the reagent is an antibody.

55. The method of claim 52 wherein the product is RNA.

56. The method of claim 55 wherein the reagent is an antisense oligonucleotide.

57. The method of claim 56 wherein the reagent is a ribozyme.

58. The method of claim 52 wherein the cell is in vitro.

59. The method of claim 52 wherein the cell is in vivo.

60. A pharmaceutical composition, comprising:
a reagent which specifically binds to a polypeptide comprising the amino acid sequence shown in SEQ ID NO: 10, 12, 13, 14, 15, 16, 17, 18, 19, 20, 21, 22, 23, 24 and 27; and
a pharmaceutically acceptable carrier.

61. The pharmaceutical composition of claim 60 wherein the reagent is an antibody.

62. A pharmaceutical composition, comprising:
a reagent which specifically binds to a product of a polynucleotide comprising the nucleotide sequence shown in SEQ ID NO: 1, 2, 3, 4, 5, 6, 7, 8, 9, 25 or 26; and
a pharmaceutically acceptable carrier.

63. The pharmaceutical composition of claim 62 wherein the reagent is a ribozyme.

64. The pharmaceutical composition of claim 62 wherein the reagent is an antisense oligonucleotide.

65. The pharmaceutical composition of claim 62 wherein the reagent is an antibody.

66. A pharmaceutical composition, comprising:
an expression vector encoding a polypeptide comprising the amino acid sequence shown in SEQ ID NO: 10, 12, 13, 14, 15, 16, 17, 18, 19, 20, 21, 22, 23, 24 and 27; and
a pharmaceutically acceptable carrier.

67. The pharmaceutical composition of claim 66 wherein the expression vector comprises SEQ ID NO: 1, 2, 3, 4, 5, 6, 7, 8, 9, 25 or 26.

68. A method of treating an epithin-like serine protease dysfunction related disease, comprising the step of:
administering to a patient in need thereof a therapeutically effective dose of a reagent that modulates a function of a human epithin-like serine protease,
whereby symptoms of the epithin-like serine protease dysfunction related disease are ameliorated.

69. The method of claim 68 wherein the reagent is identified by the method of claim 36.

70. The method of claim 68 wherein the reagent is identified by the method of claim 45.

71. The method of claim 68 wherein the reagent is identified by the method of claim 49.

72. The method of claim 68 wherein the disease is metastasis of malignant cells, tumor angiogenesis, inflammation, atherosclerosis, a neurodegenerative disease, COPD or a pathogenic infection.


SEQUENCE LISTING (27 sequences)

SEQ ID NO: 1 - DNA, 474 bp, Homo sapiens
gaattcgggaacgatggggagcaccaggcaggtggtcctcgctgtgacactgaggtgctgagccagtgagctagggtggaggggctgtgtttattggaacaaagggtggtaccaaaatgcatccccatgacccacagcccccccacacccgtccttgggtagggtacggtggggtggggtgttgggtggcctgctgctcctgttgctttcacgtagagtctcggcctgggcagtcacgtggtggtcactcctggatgtgctgtcctatccagcctctcacagctgccacccgggtatagacacctgggaagtggggccggccacagccatagccccagctagtgaccccagttagcacccaccgtccagagggctccctgcaggccaggggtcccccagcgtcaccctgttggggagagaagaaagggggttcagaggccggtacctcccctacagcagcccttgggtcattgg

SEQ ID NO: 2 - DNA, 471 bp, Homo sapiens
  misc_feature (1)...(471): n = A,T,C or G
tttgcctttttggctctggaggaatttccactccacagccgtttattgggcaacccaccgcatcccatccccgggcctcggcggcagagcaggcagaggctgcagtgggaggcaccgttccactccgggaccacgtggcgggtgtccataaatgtctgccacctttgcattgagcccattttccagatagtgaaaatgcggctcccaggggaagtcactanggtcactcctagaggggccaatgacccaagggctgctgtaggggaggtaccggcctctgaaccccctttcttctctccccaacagggtgacgctgggggacccctggcctgcagggagccctctggacggtgggtgctaactggggtcactagctggggctatggctgtggccggccccacttcccaggtgtctatacccgggtggcagctgtgagaggctggataggacagcacatccaggagtgacca

SEQ ID NO: 3 - DNA, 246 bp, Homo sapiens
agggtggagggggtcagggtgcgttttcgggcaacagttcggttccgggtgagctacagagacatgtaggggcatttggagcgtttggaactcacacgtcgaagcagtgcgccgccgacagcagccacctctctgccaccagcacggccccgcaacggtgttcccggcgccgcagccacaggtctacctgccacggccactccccacggcccgctgcgctgccgcccacaatcctggtgagcgcgg

SEQ ID NO: 4 - DNA, 181 bp, Homo sapiens
ttttttttttagggaggagggggtcagggtgcgttttcgggcaacagttcggttccgggtgagctacagagacatgtaggggcatttggagcgtttggaactcacacgtcgaagcagtgcgccgccgacagcagccacctctctgccaccagcacggccccgcaacggtgttcccggcgcc

SEQ ID NO: 5 - DNA, 446 bp, Homo sapiens
ttttttttttttttttttttttttttttttttttttttttttggggcctggaggaaattccactccacaaccgtttattgggcaacccaccccattccattcccgggccttggggggaaaaaaagcaaaggcttcagtgggaggcacccttccactccgggaccacgtggggggggtccataaatgtttgccacctttgcattgagcccattttccaaatagggaaaatgcggctcccaggggaagtcacttgggtcacttctaaaggggccaatgacccaagggcttttgtaggggaggtacccgcctttgaacccccttttttttttccccaacagggtgaacctgggggacccctggccttgcaggagccctctggacggggggtgctaactggggtcactatctggggctatggctgtggccggccccactttccaggtgtt

SEQ ID NO: 6 - DNA, 328 bp, Homo sapiens
tttttttcatttcacagttgtttattgggcaacctatcttattctattcccggtcctttttggtatatcatgcataggctgcattgggaggcaccgatccactccgggaccacgtggcgggtgtccataaaagtctgccacctttgcattgagcccattttccagatagtgaaaatgcggctcccaggggaagtcactagggtcactcctagaggggccaatgacccaaggactgctgtaggggaggtaccggcctctgaaccccctttcttctctccccaacagggtgacgctgggggacccctggcctgcagggagccctctggac

SEQ ID NO: 7 - DNA, 309 bp, Homo sapiens
gtggattccgtccagagggctccctgcaggccaggggtccccagcgtcaccctgttggggagagaagaaagggggttcagaggccggtacctcccctacagcagcccttgggtcattggcccctctaggagtgaccctagtgacttcccctgggagccgcattttcactatctggaaaatgggctcaatgcaaaggtggcagacatttatggacacccgccacgtggtcccggagtggaacggtgcctcccactgcagcctctgcctgctctgccgccgaggcccggggatgggatgcggtgggttgcc

SEQ ID NO: 8 - DNA, 345 bp, Homo sapiens
tttgcctttttggctctggaggaatttccactccacagccgtttattgggcaacccaccgcatcccatccccgggcctcggcggcagagcaggcagaggctgcagtgggaggcaccgttccactccgggaccacgtggcgggtgtccataaatgtctgccacctttgcattgagcccattttccagatagtgaaaatgcggctcccaggggaagtcactagggtcactcctagaggggccaatgacccaagggctgctgtaggggaggtaccggcctctgaaccccctttcttctctccccaacagggtgacgctgggggaccctggcctgcagggagccctctg

SEQ ID NO: 9 - DNA, 734 bp, Homo sapiens
actgtggcctggcgccggccgcgctcaccaggattgtgggcggcagcgcagcgggccgtggggagtggccgtggcaggtgagcctgtggctgcggcgccgggaacaccgttgcggggccgtgctggtggcagagaggtggctgctgtcggcggcgcactgcttcgacgtctacggggaccccaagcagtgggcggccttcctaggcacgccgttcctgagcggcgcggaggggcagctggagcgcgtggcgcgcatctacaagcacccgttctacaatctctacacgctcgactacgacgtggcgctgctggagctggcggggccggtgcgtcgcagccgcctggtgcgtcccatctgcctgcccgagcccgcgccgcgacccccggacggcacgcgctgcgtcatcaccggctggggctcggtgcgcgaaggaggctccatggcgcggcagctgcagaaggcggccgtgcgcctcctcagcgagcagacctgccgccgcttctacccagtgcagatcagcagccgcatgctgtgtgccggcttcccgcagggtggcgtggacagctgctcgggtgacgctgggggacccctggcctgcagggagccctctggacggtgggtgctaactggggtcactagctggggctatggctgtggccggccccacttcccaggtgtctatacccgggtggcagctgtgagaggctggataggacagcacatccaggagtga

SEQ ID NO: 10 - protein, 243 aa, Homo sapiens
CGLAPAALTRIVGGSAAGRGEWPWQVSLWLRRREHRCGAVLVAERWLLSAAHCFDVYGDPKQWAAFLGTPFLSGAEGQLERVARIYKHPFYNLYTLDYDVALLELAGPVRRSRLVRPICLPEPAPRPPDGTRCVITGWGSVREGGSMARQLQKAAVRLLSEQTCRRFYPVQISSRMLCAGFPQGGVDSCSGDAGGPLACREPSGRWVLTGVTSWGYGCGRPHFPGVYTRVAAVRGWIGQHIQE

SEQ ID NO: 11 - protein, 902 aa, Homo sapiens
MGSNRGRKAGGGSQDFGAGLKYDSRLENMNGFEEGVEFLPANNAKKVEKRGPRRWVVLVAVLFSFLLLSLMAGLLVWHFHYRNVRVQKVFNGHLRITNEIFLDAYENSTSTEFISLASQVKEALKLLYNEVPVLGPYHKKSAVTAFSEGSVIAYYWSEFSIPPHLAEEVDRAMAVERVVTLPPRARALKSFVLTSVVAFPIDPRMLQRTQDNSCSFALHAHGAAVTRFTTPGFPNSPYPAHARCQWVLRGDADSVLSLTFRSFDVAPCDEHGSDLVTVYDSLSPMEPHAVVRLCGTFSPSYNLTFLSSQNVFLVTLITNTGRRHLGFEATFFQLPKMSSCGGVLSDTQGTFSSPYYPGHYPPNINCTWNIKVPNNRNVKVRFKLFYLVDPNVPVGSCTKDYVEINGEKGSGERSQFVVSSNSSKITVHFHSDHSYTDTGFLAEYLSYDSNDPCPGMFMCKTGRCIRKELRCDGWADCPDYSDERYCRCNATHQFTCKNQFCKPLFWVCDSVNDCGDGSDEEGCSCPAGSFKCSNGKCLPQSQKCNGKDNCGDGSDEASCDSVNVVSCTKYTYRCQNGLCLSKGNPECDGKTDCSDGSDEKNCDCGLRSFTKQARVVGGTNADEGEWPWQVSLHALGQGHLCGASLISPDWLVSAAHCFQDDKNFKYSDYTMWTAFLGLLDQSKRSASGVQELKLKRIITHPSFNDFTFDYDIALLELEKSVEYSTVVRPICLPDATHVFPAGKAIWVTGWGHTKEGGTGALILQKGEIRVINQTTCEDLMPQQITPRMMCVGFLSGGVDSCQGDSGGPLSSAEKDGRMFQAGVVSWGEGCAQRNKPGVYTRLPCSSGLDQRAHWGIAAWTDSRPQTPTGMPDMHTWIQERNTDDIYAVASPPQHNPDCELHP

SEQ ID NO: 12 - protein, 35 aa, Homo sapiens
WVLTGVTSWGYGCGRPHFPGVYTRVAAVRGWIGQH

SEQ ID NO: 13 - protein, 18 aa, Homo sapiens
CGAVLVAERWLLSAAHCF

SEQ ID NO: 14 - protein, 17 aa, Homo sapiens
CGAVLVAERWLLSAAHC

SEQ ID NO: 15 - protein, 37 aa, Homo sapiens
GPVRRSRLVRPICLPEPAPRPPDGTRCVITGWGSVRE

SEQ ID NO: 16 - protein, 14 aa, Homo sapiens
VDSCSGDAGGPLAC

SEQ ID NO: 17 - protein, 42 aa, Homo sapiens
PLACREPSGRWVLTGVTSWGYGCGRPHFPGVYTRVAAVRGWI

SEQ ID NO: 18 - protein, 22 aa, Homo sapiens
ICLPEPAPRPPDGTRCVITGWG

SEQ ID NO: 19 - protein, 29 aa, Homo sapiens
GYGCGRPHFPGVYTRVAAVRGWIGQHIQE

SEQ ID NO: 20 - protein, 14 aa, Homo sapiens
PGVYTRVAAVRGWI

SEQ ID NO: 21 - protein, 35 aa, Homo sapiens
VRRSRLVRPICLPEPAPRPPDGTRCVITGWGSVRE

SEQ ID NO: 22 - protein, 24 aa, Homo sapiens
DSCSGDAGGPLACREPSGRWVLTG

SEQ ID NO: 23 - protein, 35 aa, Homo sapiens
AGFPQGGVDSCSGDAGGPLACREPSGRWVLTGVTS

SEQ ID NO: 24 - protein, 549 aa, Homo sapiens
MEPTVADVHLVPRTTKEVPALDAACCRAASIGVVATSLVVLTLGVLLAFLSTQGFHVDHTAELRGIRWTSSLRRETSDYHRTLTPTLEALLHFLLRPLQTLSLGLEEELLQRGIRARLREHGISLAAYGTIVSAELTGRHKGPLAERDFKSGRCPGNSFSCGNSQCVTKVNPECDDQEDCSDGSDEAHCECGLQPAWRMAGRIVGGMEASPGEFPWQASLRENKEHFCGAAIINARWLVSAAHCFNEFQDPTKWVAYVGATYLSGSEASTVRAQVVQIVKHPLYNADTADFDVAVLELTSPLPFGRHIQPVCLPAATHIFPPSKKCLISGWGYLKEDFLVKPEVLQKATVELLDQALCASLYGHSLTDRMVCAGYLDGKVDSCQGDSGGPLVCEEPSGRFFLAGIVSWGIGCAEARRPGVYARVTRLRDWILEATTKASMPLAPTMAPAPAAPSTAWPTSPESPVVSTPTKSMQALSTVPLDWVTVPKLQVKKERKYEELTYAGLKCEPSQPVAINNRAKSPRDSNCGTHLNCLQAEMTSMRIARSGDL

SEQ ID NO: 25 - DNA, 1647 bp, Homo sapiens
atggagcccactgtggctgacgtacacctcgtgcccaggacaaccaaggaagtccccgctctggatgccgcgtgctgtcgagcggccagcattggcgtggtggccaccagccttgtcgtcctcaccctgggagtccttttggccttcctctctacacagggcttccacgtggaccacacggccgagctgcggggaatccggtggaccagcagtttgcggcgggagacctcggactatcaccgcacgctgacgcccaccctggaggcactgctgcactttctgctgcgacccctccagacgctgagcctgggcctggaggaggagctattgcagcgagggatccgggcaaggctgcgggagcacggcatctccctggctgcctatggcacaattgtgtcggctgagctcacagggagacataagggacccttggcagaaagagacttcaaatcaggccgctgtccagggaactccttttcctgcgggaacagccagtgtgtgaccaaggtgaacccggagtgtgacgaccaggaggactgctccgatgggtccgacgaggcgcactgcgagtgtggcttgcagcctgcctggaggatggccggcaggatcgtgggcggcatggaagcatccccgggggagtttccgtggcaagccagccttcgagagaacaaggagcacttctgtggggccgccatcatcaacgccaggtggctggtgtctgctgctcactgcttcaatgagttccaagacccgacgaagtgggtggcctacgtgggtgcgacctacctcagcggctcggaggccagcaccgtgcgggcccaggtggtccagatcgtcaagcaccccctgtacaacgcggacacggccgactttgacgtggctgtgctggagctgaccagccctctgcctttcggccggcacatccagcccgtgtgcctcccggctgccacacacatcttcccacccagcaagaagtgcctgatctcaggctggggctacctcaaggaggacttcctggtcaagccagaggtgctgcagaaagccactgtggagctgctggaccaggcactgtgtgccagcttgtacggccattcactcactgacaggatggtgtgcgctggctacctggacgggaaggtggactcctgccagggtgactcaggaggacccctggtctgcgaggagccctctggccggttctttctggctggcatcgtgagctggggaatcgggtgtgcggaagcccggcgtccaggggtctatgcccgagtcaccaggctacgtgactggatcctggaggccaccaccaaagccagcatgcctctggcccccaccatggctcctgcccctgccgcccccagcacagcctggcccaccagtcctgagagccctgtggtcagcacccccaccaaatcgatgcaggccctcagtaccgtgcctcttgactgggtcaccgttcctaagctacaagtaaaaaaagaaagaaagtacgaggaactgacatatgcaggactgaaatgtgagccgtcacagccggtggccattaacaatagagccaagagccccagggacagcaactgtgggacacacctgaactgtctccaagcagaaatgaccagcatgcggatagccaggagtggggatctg

SEQ ID NO: 26 - DNA, 927 bp, Homo sapiens
atgttatttgagaaaagcccaaagaccaagcagcaggcccagtgtcctttcctgctgagttttcctgggataggtcccccatcacaacctactccctggggctcccagggggattcgtggagaggcggcgcccaggggggccttcgtggaggaggtgccctgggtccgcctgcccacgcgcctggctcccccgcagactgtggcctggcgccggccgcgctcaccaggattgtgggcggcagcgcagcgggccgtggggagtggccgtggcaggtgagcctgtggctgcggcgccgggaacaccgttgcggggccgtgctggtggcagagaggtggctgctgtcggcggcgcactgcttcgacgtctacggggaccccaagcagtgggcggccttcctaggcacgccgttcctgagcggcgcggaggggcagctggagcgcgtggcgcgcatctacaagcacccgttctacaatctctacacgctcgactacgacgtggcgctgctggagctggcggggccggtgcgtcgcagccgcctggtgcgtcccatctgcctgcccgagcccgcgccgcgacccccggacggcacgcgctgcgtcatcaccggctggggctcggtgcgcgaaggaggctccatggcgcggcagctgcagaaggcggccgtgcgcctcctcagcgagcagacctgccgccgcttctacccagtgcagatcagcagccgcatgctgtgtgccggcttcccgcagggtggcgtggacagctgctcgggtgacgctgggggacccctggcctgcagggagccctctggacggtgggtgctaactggggtcactagctggggctatggctgtggccggccccacttcccaggtgtctatacccgggtggcagctgtgagaggctggataggacagcacatccaggag

SEQ ID NO: 27 - protein, 309 aa, Homo sapiens
MLFEKSPKTKQQAQCPFLLSFPGIGPPSQPTPWGSQGDSWRGGAQGGLRGGGALGPPAHAPGSPADCGLAPAALTRIVGGSAAGRGEWPWQVSLWLRRREHRCGAVLVAERWLLSAAHCFDVYGDPKQWAAFLGTPFLSGAEGQLERVARIYKHPFYNLYTLDYDVALLELAGPVRRSRLVRPICLPEPAPRPPDGTRCVITGWGSVREGGSMARQLQKAAVRLLSEQTCRRFYPVQISSRMLCAGFPQGGVDSCSGDAGGPLACREPSGRWVLTGVTSWGYGCGRPHFPGVYTRVAAVRGWIGQHIQE